(19) United States
(12) Patent Application Publication
(10) Pub. No.: US 2006/0258712 A1
(43) Pub. Date: Nov. 16, 2006

(54) Methods for modulating bladder function

(75) Inventor: William Jacobson, North Wales, PA (US)

Correspondence Address: CHOATE, HALL & STEWART LLP, PATENT GROUP, TWO INTERNATIONAL PLACE, BOSTON, MA, 02110, US

(73) Assignee: Wyeth, Madison, NJ (US)

(21) Appl. No.: 11/408,323

(22) Filed: Apr. 21, 2006

Related U.S. Application Data
(60) Provisional application No. 60/674,487, filed on Apr. 24, 2005.

(51) Int. Cl. A61K 31/4433; A61K 31/381; A61K 31/343

(52) U.S. Cl. 514/337; 514/444; 514/469

(57) ABSTRACT

This invention provides methods and pharmaceutical compositions for modulating bladder function, and in particular for maintaining bladder control or treating urinary incontinence.

### CROSS REFERENCE TO RELATED APPLICATIONS

This application claims priority to U.S. Provisional Patent Application Ser. No. 60/674,487, filed Apr. 24, 2005, the entirety of which is hereby incorporated herein by reference.

### FIELD OF THE INVENTION

The present invention relates to compounds useful in modulation of bladder activity, including in the treatment, prevention, inhibition, and/or amelioration of urinary incontinence.

### BACKGROUND OF THE INVENTION

According to the American Foundation for Urologic Disease, it is estimated that over 12 million Americans suffer from urinary incontinence. At least 50% of nursing home residents are affected. Both men and women suffer from urinary incontinence, although women are disproportionately affected.

There are several different types of urinary incontinence, with the major forms being stress incontinence, urge incontinence, and mixed incontinence. Stress incontinence refers to urinary leakage that occurs during physical activity, such as coughing, sneezing, or laughing, that stresses the abdomen. Urge incontinence (also known as bladder instability, neurogenic bladder, voiding dysfunction, hyperactive bladder or detrusor overactivity) results from an overactive bladder contracting involuntarily, resulting in urine loss and an urgent need to void. Urge incontinence is often caused by inappropriate action of the nerves that normally control the bladder. Mixed incontinence is a combination of stress incontinence and urge incontinence.

Available treatments for urinary incontinence are dismal, ranging from absorbent pads, to mechanical devices that restrict urine flow, to devices that apply electric shocks to various muscles. Efforts have been made to develop effective pharmacological interventions, but thus far have met with limited success. One promising new area of investigation centers upon recent findings that serotonin signaling pathways are involved in controlling muscles involved in urine release. Specifically, it has been reported that agonists of the 5HT_{2C} serotonin receptor can be effective inhibitors of urinary incontinence. See United States Patent Application 2004/0235856 by McMurray et al, the entire contents of which are incorporated herein by reference. There remains a need for the identification of potent, specific 5HT_{2C} inhibitors that are effective inhibitors of urinary incontinence.

### SUMMARY OF THE INVENTION

The present invention provides methods for modulating activity of bladder tissues in a mammal, particularly including methods for maintaining urinary bladder control. In particular, according to the present invention, compounds of formula I: 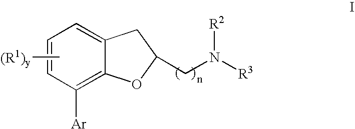
or a pharmaceutically acceptable salt thereof, wherein:
n is one or two;
each of R² and R³ is independently hydrogen, methyl, ethyl, 2-fluoroethyl, 2,2-difluoroethyl or cyclopropyl;
each R¹ is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} substituents;
each R^{x} is independently selected from halogen, OH, lower alkyl, lower alkoxy, lower haloalkyl, lower haloalkoxy, or CN; and
y is 0-3,
   which are highly specific agonists of the 5HT_{2C} receptor, are useful in the treatment of urinary incontinence.

The present invention provides, among other things, methods of treating urinary incontinence by administrating to an individual in need thereof a pharmaceutically effective amount of a compound of formula I. The invention also provides pharmaceutical compositions of compounds of formula I formulated and dosed for treatment of urinary incontinence, as well as combinations of compounds of formula I with one or more other agents useful in the treatment of urinary incontinence and/or other disorders or diseases suffered by individuals with urinary incontinence. Yet other aspects of the present invention will be clear to those of ordinary skill in the art upon review of the present specification and claims.

### DETAILED DESCRIPTION OF CERTAIN EMBODIMENTS OF THE INVENTION

1. Compounds

Compounds useful for modulating bladder function, and in particular treating urinary incontinence according to the present invention include compounds of formula I 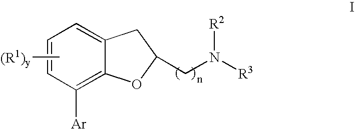
or a pharmaceutically acceptable salt thereof, wherein:
n is one or two;
each of R² and R³ is independently hydrogen, methyl, ethyl, 2-fluoroethyl, 2,2-difluoroethyl or cyclopropyl;
each R¹ is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} substituents;
each R^{x} is independently selected from halogen, OH, lower alkyl, lower alkoxy, lower haloalkyl, lower haloalkoxy, or CN; and
y is 0-3.

The term “lower alkyl,” as used herein, refers to a hydrocarbon chain having up to 4 carbon atoms, preferably 1 to 3 carbon atoms, and more preferably I to 2 carbon atoms. The term “alkyl” includes, but is not limited to, straight and branched chains such as methyl, ethyl, n-propyl, isopropyl, n-butyl, isobutyl, sec-butyl, or t-butyl.

The term “alkoxy,” as used herein, refers to the group —OR, wherein R is a lower alkyl group.

The terms “halogen” or “halo,” as used herein, refer to chlorine, bromine, fluorine or iodine.

The term “haloalkyl,” as used herein, or as part of a moiety such as “haloalkoxy” refers to an alkyl group, as defined herein, that has one or more halogen substituents. In certain embodiment, every hydrogen atom on said alkyl group is replaced by a halogen atom. Such haloalkyl groups include —CF₃. Such haloalkoxy groups include —OCF₃.

It will be appreciated by those of ordinary skill in the art that reference to a compound herein is intended to include reference to any and all related forms such as stereoisomers, polymorphs, hydrates, etc. Also, compounds may be provided as pro-drugs or other forms converted into the active agent during manufacture, processing, formulation, delivery, or in the body.

The term “pharmaceutically acceptable salts” or “pharmaceutically acceptable salt” refers to salts derived from treating a compound of formula I with an organic or inorganic acid such as, for example, acetic, lactic, citric, cinnamic, tartaric, succinic, fumaric, maleic, malonic, mandelic, malic, oxalic, propionic, hydrochloric, hydrobromic, phosphoric, nitric, sulfuric, glycolic, pyruvic, methanesulfonic, ethanesulfonic, toluenesulfonic, salicylic, benzoic, or similarly known acceptable acids. In certain embodiments, the present invention provides the hydrochloride salt of a compound of formula I.

As defined generally above, each of the R² and R³ groups of formula I is independently hydrogen, methyl, ethyl, 2-fluoroethyl, 2,2-difluoroethyl or cyclopropyl. In certain embodiments, one of the R² and R³ groups of formula I is hydrogen and the other R² or R³ group of formula I is hydrogen, methyl, ethyl, 2-fluoroethyl, 2,2-difluoroethyl or cyclopropyl. In other embodiments, neither of the R² and R³ groups of formula I is hydrogen. In still other embodiments, both of the R² and R³ groups of formula I are hydrogen.

As defined generally above, each R¹ group of formula I is independently hydrogen, halogen, OH, lower alkyl, lower alkoxy, lower haloalkyl, lower haloalkoxy, or CN. In certain embodiments, each R¹ group of formula I is hydrogen. In other embodiments, at least one of R¹ group of formula I is halogen. In still other embodiments, y is I and R¹ is halogen.

According to another embodiment, y is 1 and R¹ is at the 5-position of the dihydrobenzofuran ring of formula I, thus forming a compound of formula Ia: 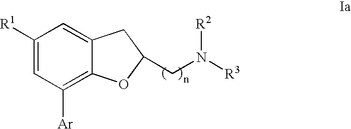
or a pharmaceutically acceptable salt thereof, wherein each of R¹, R², R³, Ar, and n are as defined above for compounds of formula I and in classes and subclasses as described above and herein.

According to yet another embodiment, y is 1 and R¹ is at the 6-position of the dihydrobenzofuran ring of formula I, thus forming a compound of formula Ia′: 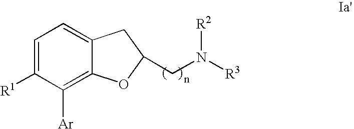
or a pharmaceutically acceptable salt thereof, wherein each of R¹, R², R³, Ar, and n are as defined above for compounds of formula I and in classes and subclasses as described above and herein.

As defined generally above, the Ar group of formula I is thienyl, furyl, pyridyl, or phenyl, wherein Ar is optionally substituted with one or more substituents independently selected from halogen, OH, lower alkyl, lower alkoxy, haloalkyl, haloalkoxy, or CN. In certain embodiments, the Ar group of formula I is unsubstituted phenyl. In other embodiments, the Ar group of formula I is phenyl with at least one substituent in the ortho position. In other embodiments, the Ar group of formula I is phenyl with at least one substituent in the ortho position selected from halogen, lower alkyl, lower alkoxy, or trifluoromethyl. According to another aspect the present invention provides a compound of formula I wherein Ar is phenyl di-substituted in the ortho and meta positions with independently selected halogen lower alkyl, or lower alkoxy. Yet another aspect of the present invention provides a compound of formula I wherein Ar is phenyl di-substituted in the ortho and para positions with independently selected halogen lower alkyl, or lower alkoxy. In other embodiment, the present invention provides a compound of formula I wherein Ar is phenyl di-substituted in the ortho positions with independently selected halogen lower alkyl, or lower alkoxy. Exemplary substituents on the phenyl moiety of the Ar group of formula I include OMe, fluoro, chloro, methyl, and trifluoromethyl.

In certain embodiments, the present invention provides a compound of formula Ia′ wherein Ar is phenyl with one substituent in the ortho position selected from halogen, lower alkyl, lower alkoxy, or trifluoromethyl.

According to one embodiment, Ar is phenyl substituted with one R^{x} substituent in the ortho-position, thus forming a compound of formula Ib, or with an R^{x} substituent in both ortho-positions, thus forming a compound of formula Ic: 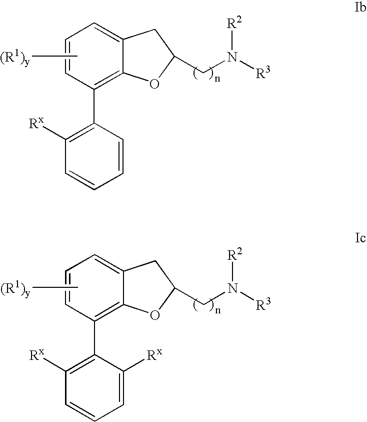
or a pharmaceutically acceptable salt thereof, wherein each R¹, R², R³, R^{x}, y and n are as defined above for compounds of formula I and in classes and subclasses as described above and herein.

In certain embodiments, the Ar group of formula I is selected from the following:

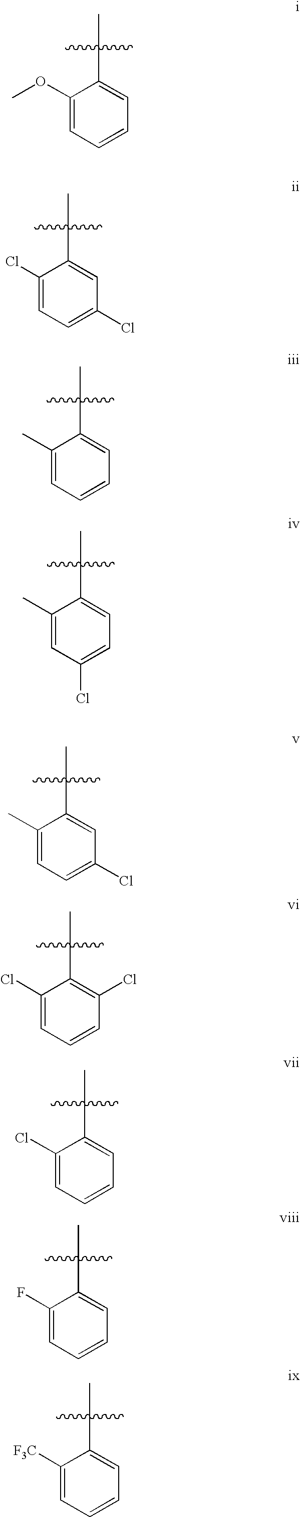

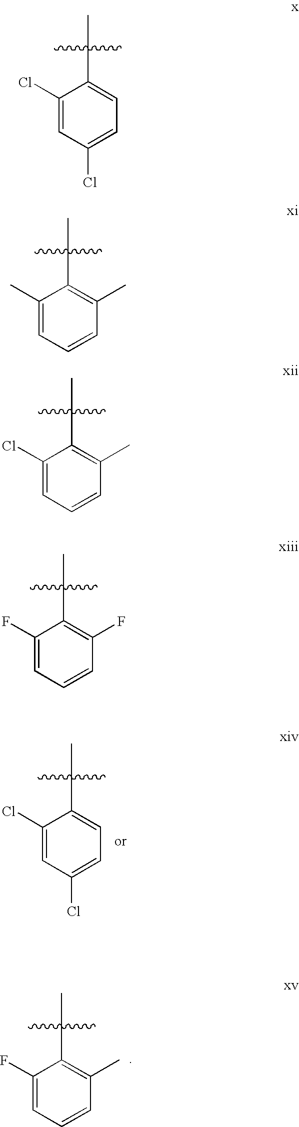

According to yet another embodiment, the present invention provides a compound of formula Id or Ie: 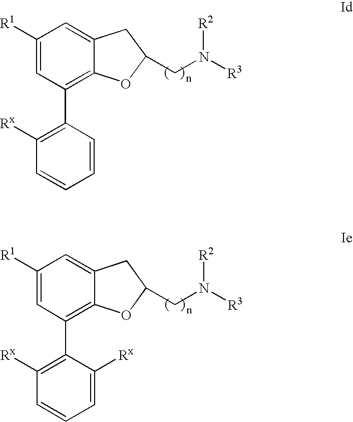
or a pharmaceutically acceptable salt thereof, wherein each R¹, R², R³, R^{x}, y and n are as defined above for compounds of formula I and in classes and subclasses as described above and herein.

According to another embodiment, the present invention provides a compound of formula If or Ig: 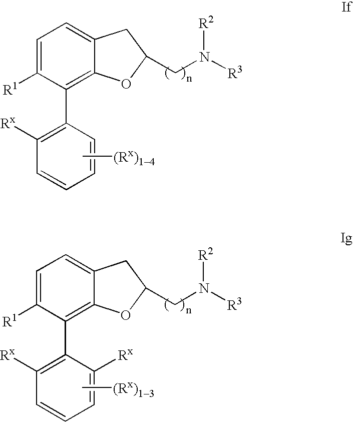
or a pharmaceutically acceptable salt thereof, wherein each R¹, R², R³, R^{x}, and n are as defined above for compounds of formula I and in classes and subclasses as described above and herein.

In certain embodiments, the present invention provides a compound of formula Ih or Ii: 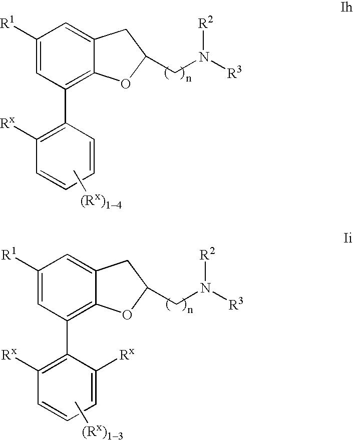
or a pharmaceutically acceptable salt thereof, wherein each R¹, R², R³, R^{x}, and n are as defined above for compounds of formula I and in classes and subclasses as described above and herein.

Compounds of the present invention contain asymmetric carbon atoms and thus give rise to stereoisomers, including enantiomers and diastereomers. Accordingly, it is contemplated that the present invention relates to all of these stereoisomers, as well as to mixtures of the stereoisomers. Throughout this application, the name of the product of this invention, where the absolute configuration of an asymmetric center is not indicated, is intended to embrace the individual stereoisomers as well as mixtures of stereoisomers. In certain embodiments of the invention, compounds having an absolute (R) configuration are preferred.

In certain embodiments, the present invention provides a compound of formula VIa or VIb: 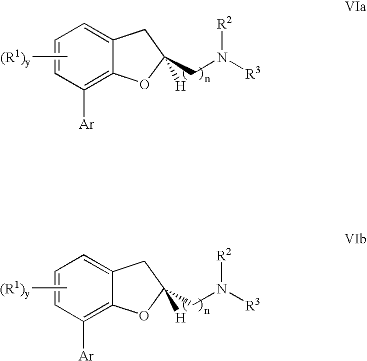
or a pharmaceutically acceptable salt thereof, wherein each R¹, R², R³, R^{x}, y and n are as defined above for compounds of formula I and in classes and subclasses as described above and herein.

According to another embodiment, the present invention provides a compound of formula VIc or VId: 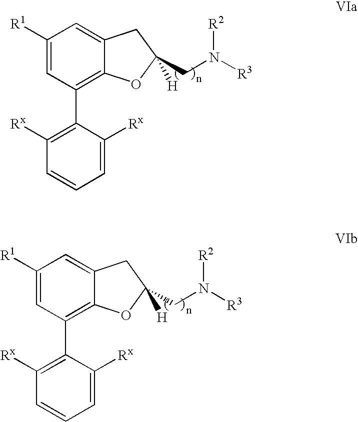
or a pharmaceutically acceptable salt thereof, wherein each R¹, R², R³, R^{x}, y and n are as defined above for compounds of formula I and in classes and subclasses as described above and herein.

Where an enantiomer is preferred, it may, in some embodiments be provided substantially free of the corresponding enantiomer. Thus, an enantiomer substantially free of the corresponding enantiomer refers to a compound which is isolated or separated via separation techniques or prepared free of the corresponding enantiomer. “Substantially free,” as used herein, means that the compound is made up of a significantly greater proportion of one enantiomer. In certain embodiments the compound is made up of at least about 90% by weight of a preferred enantiomer. In other embodiments of the invention, the compound is made up of at least about 99% by weight of a preferred enantiomer. Preferred enantiomers may be isolated from racemic mixtures by any method known to those skilled in the art, including chiral high pressure liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

It is further recognized that atropisomers of the present compounds may exit. The present invention thus encompasses atropisomeric forms of compounds of formula I as defined above, and in classes and sublcasses described above and herein.

Exemplary compounds useful for the methods of the present invention are set forth in Table 1, below.

TABLE 1
Exemplary Compounds of Formula I

(±)-1-{7-[3,5-bis(trifluoromethyl)phenyl]-2,3-dihydro-1-benzofuran-2-yl}methanamine,
(±)-1-[7-(3-chloro-4-fluorophenyl)-2,3-dihydro-1-benzofuran-2-yl]methanamine,
(±)-1-[7-(3,5-dichlorophenyl)-2,3-dihydro-1-benzofuran-2-yl]methanamine,
(±)-1-(7-phenyl-2,3-dihydro-1-benzofuran-2-yl)methanamine,
(+)-(1-(7-phenyl-2,3-dihydro-1-benzofuran-2-yl)methanamine,
(−)-1-(7-phenyl-2,3-dihydro-1-benzofuran-2-yl)methanamine,
(±)-1-[7-(3-methylphenyl)-2,3-dihydro-1-benzofuran-2-yl]methanamine,
(+)-1-[7-(3-methylphenyl)-2,3-dihydro-1-benzofuran-2-yl]methanamine,
(−)-1-[7-(3-methylphenyl)-2,3-dihydro-1-benzofuran-2-yl]methanamine,
(±)-1-(7-thien-3-yl-2,3-dihydro-1-benzofuran-2-yl)methanamine,
(+)-1-(7-thien-3-yl-2,3-dihydro-1-benzofuran-2-yl)methanamine,
(−)-1-(7-thien-3-yl-2,3-dihydro-1-benzofuran-2-yl)methanamine,
(±)-1-[7-(2-methylphenyl)-2,3-dihydro-1-benzofuran-2-yl]methanamine,
(+)-1-[7-(2-methylphenyl)-2,3-dihydro-1-benzofuran-2-yl]methanamine,
(−)-1-[7-(2-methylphenyl)-2,3-dihydro-1-benzofuran-2-yl]methanamine,
(±)-1-[7-(2-fluorophenyl)-2,3-dihydro-1-benzofuran-2-yl]methanamine,
(+)-1-[7-(2-fluorophenyl)-2,3-dihydro-1-benzofuran-2-yl]methanamine,
(−)-1-[7-(2-fluorophenyl)-2,3-dihydro-1-benzofuran-2-yl]methanamine,
(±)-1-{7-[2-(trifluoromethyl)phenyl]-2,3-dihydro-1-benzofuran-2-yl}methanamine,
(−)-1-{7-[2-(trifluoromethyl)phenyl]-2,3-dihydro-1-benzofuran-2-yl}methanamine,
(+)-1-{7-[2-(trifluoromethyl)phenyl]-2,3-dihydro-1-benzofuran-2-yl}methanamine,
(±)-1-[7-(2,6-dimethylphenyl)-2,3-dihydro-1-benzofuran-2-yl]methanamine,
(−)-1-[7-(2,6-dimethylphenyl)-2,3-dihydro-1-benzofuran-2-yl]methanamine,
(+)-1-[7-(2,6-dimethylphenyl)-2,3-dihydro-1-benzofuran-2-yl]methanamine,
(±)-1-[7-(2-methoxyphenyl)-2,3-dihydro-1-benzofuran-2-yl]methanamine,
(±)-1-[7-(2-chlorophenyl)-2,3-dihydro-1-benzofuran-2-yl]methanamine,
(±)-1-[7-(3-fluorophenyl)-2,3-dihydro-1-benzofuran-2-yl]methanamine,
(±)-1-[7-(3-chlorophenyl)-2,3-dihydro-1-benzofuran-2-yl]methanamine,
(+)-1-[7-(3-chlorophenyl)-2,3-dihydro-1-benzofuran-2-yl]methanamine,
(−)-1-[7-(3-chlorophenyl)-2,3-dihydro-1-benzofuran-2-yl]methanamine,
(±)-1-[7-(3-methoxyphenyl)-2,3-dihydro-1-benzofuran-2-yl]methanamine,
(±)-1-{7-[3-(trifluoromethyl)phenyl]-2,3-dihydro-1-benzofuran-2-yl}methanamine,
(±)-1-[7-(4-methylphenyl)-2,3-dihydro-1-benzofuran-2-yl]methanamine,
(+)-1-[7-(4-methylphenyl)-2,3-dihydro-1-benzofuran-2-yl]methanamine,
(−)-1-[7-(4-methylphenyl)-2,3-dihydro-1-benzofuran-2-yl]methanamine,
(±)-1-[7-(4-fluorophenyl)-2,3-dihydro-1-benzofuran-2-yl]methanamine,
(+)-1-[7-(4-fluorophenyl)-2,3-dihydro-1-benzofuran-2-yl]methanamine,
(−)-1-[7-(4-fluorophenyl)-2,3-dihydro-1-benzofuran-2-yl]methanamine,
(±)-1-[7-(4-chlorophenyl)-2,3-dihydro-1-benzofuran-2-yl]methanamine,
(+)-1-[7-(4-chlorophenyl)-2,3-dihydro-1-benzofuran-2-yl]methanamine,
(−)-1-[7-(4-chlorophenyl)-2,3-dihydro-1-benzofuran-2-yl]methanamine,
(±)-1-[7-(4-methoxyphenyl)-2,3-dihydro-1-benzofuran-2-yl]methanamine,
(+)-1-[7-(4-methoxyphenyl)-2,3-dihydro-1-benzofuran-2-yl]methanamine,
(−)-1-[7-(4-methoxyphenyl)-2,3-dihydro-1-benzofuran-2-yl]methanamine,
(±)-1-{7-[4-(trifluoromethyl)phenyl]-2,3-dihydro-1-benzofuran-2-yl}methanamine,
(±)-1-[7-(2,4-dichlorophenyl)-2,3-dihydro-1-benzofuran-2-yl]methanamine,
(±)-1-(5-chloro-7-phenyl-2,3-dihydro-1-benzofuran-2-yl)methanamine,
(+)-1-(5-chloro-7-phenyl-2,3-dihydro-1-benzofuran-2-yl)methanamine,
(−)-1-(5-chloro-7-phenyl-2,3-dihydro-1-benzofuran-2-yl)methanamine,
(±)-1-[5-chloro-7-(2-chlorophenyl)-2,3-dihydro-1-benzofuran-2-yl]methanamine,
(±)-1-[5-chloro-7-(3-methylphenyl)-2,3-dihydro-1-benzofuran-2-yl]methanamine,
(±)-1-[5-chloro-7-(3-chlorophenyl)-2,3-dihydro-1-benzofuran-2-yl]methanamine,
(±)-(5-chloro-7-thien-3-yl-2,3-dihydro-1-benzofuran-2-yl)methylamine,
(−)-(5-chloro-7-thien-3-yl-2,3-dihydro-1-benzofuran-2-yl)methylamine,
(+)-(5-chloro-7-thien-3-yl-2,3-dihydro-1-benzofuran-2-yl)methylamine,
(+)-N-[(5-chloro-7-thien-3-yl-2,3-dihydro-1-benzofuran-2-yl)methyl]-N-methylamine,
(−)-N-[(5-chloro-7-thien-3-yl-2,3-dihydro-1-benzofuran-2-yl)methyl]-N-methylamine,
(±)-1-[5-chloro-7-(2-methylphenyl)-2,3-dihydro-1-benzofuran-2-yl]methanamine,
(+)-1-[5-chloro-7-(2-methylphenyl)-2,3-dihydro-1-benzofuran-2-yl]methanamine,
(−)-1-[5-chloro-7-(2-methylphenyl)-2,3-dihydro-1-benzofuran-2-yl]methanamine,
(±)-1-(4-fluoro-7-phenyl-2,3-dihydro-1-benzofuran-2-yl)methanamine,
(±)-1-[4-fluoro-7-(2-methylphenyl)-2,3-dihydro-1-benzofuran-2-yl]methanamine,
(±)-1-[7-(2-chlorophenyl)-5-fluoro-2,3-dihydro-1-benzofuran-2-yl]methanamine,
(+)-1-[7-(2-chlorophenyl)-5-fluoro-2,3-dihydro-1-benzofuran-2-yl]methanamine,
(−)-1-[7-(2-chlorophenyl)-5-fluoro-2,3-dihydro-1-benzofuran-2-yl]methanamine,
(±)-1-(5-fluoro-7-phenyl-2,3-dihydro-1-benzofuran-2-yl)methanamine,
(±)-1-[5-fluoro-7-(2-methylphenyl)-2,3-dihydro-1-benzofuran-2-yl]methanamine,
(+)-1-[5-fluoro-7-(2-methylphenyl)-2,3-dihydro-1-benzofuran-2-yl]methanamine,
(−)-1-[5-fluoro-7-(2-methylphenyl)-2,3-dihydro-1-benzofuran-2-yl]methanamine,
(±)-1-[5-fluoro-7-(2-fluorophenyl)-2,3-dihydro-1-benzofuran-2-yl]methanamine,
(±)-1-{5-fluoro-7-[2-(trifluoromethyl)phenyl]-2,3-dihydro-1-benzofuran-2-yl}methanamine,
(±)-(4,5-difluoro-7-phenyl-2,3-dihydro-1-benzofuran-2-yl)methylamine,
(±)-1-[4,5-difluoro-7-(2-methylphenyl)-2,3-dihydro-1-benzofuran-2-yl]methanamine,
(±)-1-(5-chloro-2-methyl-7-phenyl-2,3-dihydro-1-benzofuran-2-yl)methanamine,
(±)-(5-chloro-2-methyl-7-thien-3-yl-2,3-dihydro-1-benzofuran-2-yl)methylamine,
(±)-(5-chloro-2-methyl-7-thien-2-yl-2,3-dihydro-1-benzofuran-2-yl)methylamine,
(+)-1-[7-(2-methoxyphenyl)-2,3-dihydro-1-benzofuran-2-yl]methanamine,
(−)-1-[7-(2-chlorophenyl)-2,3-dihydro-1-benzofuran-2-yl]methanamine,
(+)-1-[7-(2-chlorophenyl)-2,3-dihydro-1-benzofuran-2-yl]methanamine,
(+)-1-[7-(3-fluorophenyl)-2,3-dihydro-1-benzofuran-2-yl]methanamine,
(−)-1-[7-(3-fluorophenyl)-2,3-dihydro-1-benzofuran-2-yl]methanamine,
(+)-1-[7-(3-methoxyphenyl)-2,3-dihydro-1-benzofuran-2-yl]methanamine,
(−)-1-[7-(3-methoxyphenyl)-2,3-dihydro-1-benzofuran-2-yl]methanamine,
(−)-1-{7-[3-(trifluoromethyl)phenyl]-2,3-dihydro-1-benzofuran-2-yl}methanamine,
(+)-1-{7-[3-(trifluoromethyl)phenyl]-2,3-dihydro-1-benzofuran-2-yl}methanamine,
(+)-1-{7-[4-(trifluoromethyl)phenyl]-2,3-dihydro-1-benzofuran-2-yl}methanamine,
(−)-1-{7-[4-(trifluoromethyl)phenyl]-2,3-dihydro-1-benzofuran-2-yl}methanamine,
(±)-1-[7-(2,6-difluorophenyl)-2,3-dihydro-1-benzofuran-2-yl]methanamine,
(±)-1-[7-(2,6-dichlorophenyl)-2,3-dihydro-1-benzofuran-2-yl]methanamine,
(−)-1-[7-(2,6-dichlorophenyl)-2,3-dihydro-1-benzofuran-2-yl]methanamine,
(+)-1-[7-(2,6-dichlorophenyl)-2,3-dihydro-1-benzofuran-2-yl]methanamine,
(±)-1-[7-(2,4-dimethoxyphenyl)-2,3-dihydro-1-benzofuran-2-yl]methanamine,
(−)-1-[7-(2,4-dimethoxyphenyl)-2,3-dihydro-1-benzofuran-2-yl]methanamine,
(+)-1-[7-(2,4-dimethoxyphenyl)-2,3-dihydro-1-benzofuran-2-yl]methanamine,
(±)-1-[7-(2,4-difluorophenyl)-2,3-dihydro-1-benzofuran-2-yl]methanamine,
(+)-1-[7-(2,4-difluorophenyl)-2,3-dihydro-1-benzofuran-2-yl]methanamine,
(−)-1-[7-(2,4-difluorophenyl)-2,3-dihydro-1-benzofuran-2-yl]methanamine,
(−)-1-[7-(2,4-dichlorophenyl)-2,3-dihydro-1-benzofuran-2-yl]methanamine,
(+)-1-[7-(2,4-dichlorophenyl)-2,3-dihydro-1-benzofuran-2-yl]methanamine,
(−)-1-{5-fluoro-7-[2-(trifluoromethyl)phenyl]-2,3-dihydro-1-benzofuran-2-yl}methanamine,
(+)-1-{5-fluoro-7-[2-(trifluoromethyl)phenyl]-2,3-dihydro-1-benzofuran-2-yl}methanamine,
(±)-1-[7-(2,3-dimethylphenyl)-2,3-dihydro-1-benzofuran-2-yl]methanamine,
(±)-{[7-(2,3-dimethoxyphenyl)-2,3-dihydro-1-benzofuran-2-yl]methyl}amine,
(−)-{[(7-(2,3-dimethoxyphenyl)-2,3-dihydro-1-benzofuran-2-yl]methyl}amine,
(+)-{[(7-(2,3-dimethoxyphenyl)-2,3-dihydro-1-benzofuran-2-yl]methyl}amine,
(+)-{[7-(4-chloro-2-methylphenyl)-2,3-dihydro-1-benzofuran-2-yl]methyl}amine,
(−)-{[7-(4-chloro-2-methylphenyl)-2,3-dihydro-1-benzofuran-2-yl]methyl}amine,
(±)-{[7-(2,3-difluorophenyl)-2,3-dihydro-1-benzofuran-2-yl]methyl}amine,
(±)-{[7-(2,5-dimethylphenyl)-2,3-dihydro-1-benzofuran-2-yl]methyl}amine,
(±)-{[7-(2,5-dimethoxyphenyl)-2,3-dihydro-1-benzofuran-2-yl]methyl}amine,
(±)-{[7-(2,5-dichlorophenyl)-2,3-dihydro-1-benzofuran-2-yl]methyl}amine,
(+)-{[7-(2,5-dichlorophenyl)-2,3-dihydro-1-benzofuran-2-yl]methyl}amine,
(−)-{[7-(2,5-dichlorophenyl)-2,3-dihydro-1-benzofuran--yl]methyl}amine,
(±)-{[7-(2,4,6-trichlorophenyl)-2,3-dihydro-1-benzofuran-2-yl]methyl}amine,
(±)-{[7-(4-chloro-2-methylphenyl)-2,3-dihydro-1-benzofuran-2-yl]methyl}amine,
(±)-{[7-(5-chloro-2-methylphenyl)-2,3-dihydro-1-benzofuran-2-yl]methyl}amine,
(±)-{[7-(5-chloro-2-methoxyphenyl)-2,3-dihydro-1-benzofuran-2-yl]methyl}amine,
(±)-[(7-pyridin-3-yl-2,3-dihydro-1-benzofuran-2-yl)methyl]amine,
(+)-{[7-pyridin-3-yl-2,3-dihydro-1-benzofuran-2-yl]methyl}amine,
(−)-{[7-pyridin-3-yl-2,3-dihydro-1-benzofuran-2-yl]methyl}amine,
(±)-{[5-fluoro-7-(2-methoxyphenyl)-2,3-dihydro-1-benzofuran-2-yl]methyl}amine,
(±)-{[5-fluoro-7-(3-fluorophenyl)-2,3-dihydro-1-benzofuran-2-yl]methyl}amine,
(±)-{[5-fluoro-7-(3-methoxyphenyl)-2,3-dihydro-1-benzofuran-2-yl]methyl}amine,
(±)-{[5-fluoro-7-(3-methylphenyl)-2,3-dihydro-1-benzofuran-2-yl]methyl}amine,
(±)-{[5-fluoro-7-(4-fluorophenyl)-2,3-dihydro-1-benzofuran-2-yl]methyl}amine,
(±)-{[5-fluoro-7-(4-chlorophenyl)-2,3-dihydro-1-benzofuran-2-yl]methyl}amine,
(±)-{[5-fluoro-7-(4-methylphenyl)-2,3-dihydro-1-benzofuran-2-yl]methyl}amine,
(±)-{[5-fluoro-7-(4-methoxyphenyl)-2,3-dihydro-1-benzofuran-2-yl]methyl}amine,
(±)-[(5-fluoro-7-thien-3-yl-2,3-dihydro-1-benzofuran-2-yl)methyl]amine,
(±)-{[5-fluoro-7-(3-furyl)-2,3-dihydro-1-benzofuran-2-yl]methyl}amine,
(±)-[(5-fluoro-7-pyridin-2-yl-2,3-dihydro-1-benzofuran-2-yl)methyl]amine,
(±)-[(5-fluoro-7-pyridin-3-yl-2,3-dihydro-1-benzofuran-2-yl)methyl]amine,
(−)-[(5-fluoro-7-pyridin-3-yl-2,3-dihydro-1-benzofuran-2-yl)methyl]amine,
(+)-{[5-fluoro-7-pyridin-3-yl-2,3-dihydro-1-benzofuran-2-yl]methyl}amine,
(±)-[(5-fluoro-7-pyridin-4-yl-2,3-dihydro-1-benzofuran-2-yl)methyl]amine,
(±)-[(5-fluoro-7-pyrimidin-5-yl-2,3-dihydro-1-benzofuran-2-yl)methyl]amine,
(±)-{[7-(2,3-dichlorophenyl)-5-fluoro-2,3-dihydro-1-benzofuran-2-yl]methyl}amine,
(±)-{[7-(2,3-dimethoxyphenyl)-5-fluoro-2,3-dihydro-1-benzofuran-2-yl]methyl}amine,
(−)-{[7-(2,3-dimethoxyphenyl)-5-fluoro-2,3-dihydro-1-benzofuran-2-yl]methyl}amine,
(+)-{[7-(2,3-dimethoxyphenyl)-5-fluoro-2,3-dihydro-1-benzofuran-2-yl]methyl}amine,
(±)-{[7-(2,4-difluorophenyl)-5-fluoro-2,3-dihydro-1-benzofuran-2-yl]methyl}amine,
(±)-{[7-(2,4-dichlorophenyl)-5-fluoro-2,3-dihydro-1-benzofuran-2-yl]methyl}amine,
(−)-{[5-fluoro-7-(2,4-dichlorophenyl)-2,3-dihydro-1-benzofuran-2-yl]methyl}amine,
(+)-{[5-fluoro-7-(2,4-dichlorophenyl)-2,3-dihydro-1-benzofuran-2-yl]methyl}amine,
(±)-{[7-(2,4-dimethoxyphenyl)-5-fluoro-2,3-dihydro-1-benzofuran-2-yl]methyl}amine,
(±)-{[7-(2,5-difluorophenyl)-5-fluoro-2,3-dihydro-1-benzofuran-2-yl]methyl}amine,
(±)-{[7-(2,5-dichlorophenyl)-5-fluoro-2,3-dihydro-1-benzofuran-2-yl]methyl}amine,
(±)-{[7-(2,5-dimethylphenyl)-5-fluoro-2,3-dihydro-1-benzofuran-2-yl]methyl}amine,
(±)-{[7-(2,5-dimethoxyphenyl)-5-fluoro-2,3-dihydro-1-benzofuran-2-yl]methyl}amine,
(±)-{[5-fluoro-7-(5-methoxy-2-methylphenyl)-2,3-dihydro-1-benzofuran-2-yl]methyl}amine,
(±)-{[5-fluoro-7-(2-methoxy-5-methylphenyl)-2,3-dihydro-1-benzofuran-2-yl]methyl}amine,
(±)-{[7-(2,6-difluorophenyl)-5-fluoro-2,3-dihydro-1-benzofuran-2-yl]methyl}amine,
(±)-{[7-(2,6-dimethylphenyl)-5-fluoro-2,3-dihydro-1-benzofuran-2-yl]methyl}amine,
(+)-{[7-(2,6-dimethylphenyl)-5-fluoro-2,3-dihydro-1-benzofuran-2-yl]methyl}amine,
(−)-{[7-(2,6-dimethylphenyl)-5-fluoro-2,3-dihydro-1-benzofuran-2-yl]methyl}amine,
(±)-{[7-(2,6-dichlorophenyl)-5-fluoro-2,3-dihydro-1-benzofuran-2-yl]methyl}amine,
(±)-N-{[7-(2,6-dichlorophenyl)-5-fluoro-2,3-dihydro-1-benzofuran-2-
yl]methyl}cyclopropanamine,
(±)-1-cyclopropyl-N-{[7-(2,6-dichlorophenyl)-5-fluoro-2,3-dihydro-1-benzofuran-2-
yl]methyl}methanamine,
(±)-N-{[7-(2,6-dichlorophenyl)-5-fluoro-2,3-dihydro-1-benzofuran-2-yl]methyl}ethanamine,
(±)-{[7-(2,6-dichlorophenyl)-5-fluoro-2,3-dihydro-1-benzofuran-2-yl]methyl}dimethylamine,
(±)-{[5-chloro-7-(2-fluorophenyl)-2,3-dihydro-1-benzofuran-2-yl]methyl}amine,
(±)-{[5-chloro-7-(2-methoxyphenyl)-2,3-dihydro-1-benzofuran-2-yl]methyl}amine,
(±)-{[5-chloro-7-(3-furyl)-2,3-dihydro-1-benzofuran-2-yl]methyl}amine,
(±)-{[5-chloro-7-(2,3-difluorophenyl)-2,3-dihydro-1-benzofuran-2-yl]methyl}amine,
(−)-{[5-chloro-7-(2,3-difluorophenyl)-2,3-dihydro-1-benzofuran-2-yl]methyl}amine,
(+)-{[5-chloro-7-(2,3-difluorophenyl)-2,3-dihydro-1-benzofuran-2-yl]methyl}amine,
(±)-{[5-chloro-7-(2,3-dichlorophenyl)-2,3-dihydro-1-benzofuran-2-yl]methyl}amine,
(±)-{[5-chloro-7-(2,3-dimethylphenyl)-2,3-dihydro-1-benzofuran-2-yl]methyl}amine,
(±)-{[5-chloro-7-(2,3-dimethoxyphenyl)-2,3-dihydro-1-benzofuran-2-yl]methyl}amine,
(±)-{[5-chloro-7-(2,4-difluorophenyl)-2,3-dihydro-1-benzofuran-2-yl]methyl}amine,
(±)-{[5-chloro-7-(2,4-dichlorophenyl)-2,3-dihydro-1-benzofuran-2-yl]methyl}amine,
(−)-{[5-chloro-7-(2,4-dichlorophenyl)-2,3-dihydro-1-benzofuran-2-yl]methyl}amine,
(+)-{[5-chloro-7-(2,4-dichlorophenyl)-2,3-dihydro-1-benzofuran-2-yl]methyl}amine,
(±)-{[5-chloro-7-(2,4-dimethoxyphenyl)-2,3-dihydro-1-benzofuran-2-yl]methyl}amine,
(±)-{[5-chloro-7-(2,5-difluorophenyl)-2,3-dihydro-1-benzofuran-2-yl]methyl}amine,
(±)-{[5-chloro-7-(2,5-dichlorophenyl)-2,3-dihydro-1-benzofuran-2-yl]methyl}amine,
(±)-{[5-chloro-7-(5-chloro-2-methoxyphenyl)-2,3-dihydro-1-benzofuran-2-yl]methyl}amine,
(±)-{[5-chloro-7-(3,4-difluorophenyl)-2,3-dihydro-1-benzofuran-2-yl]methyl}amine,
(±)-{[5-chloro-7-(3-chloro-4-fluorophenyl)-2,3-dihydro-1-benzofuran-2-yl]methyl}amine,
(±)-{[5-chloro-7-(2,6-dimethylphenyl)-2,3-dihydro-1-benzofuran-2-yl]methyl}amine,
(−)-{[5-chloro-7-(2,6-dimethylphenyl)-2,3-dihydro-1-benzofuran-2-yl]methyl}amine,
(+)-{[5-chloro-7-(2,6-dimethylphenyl)-2,3-dihydro-1-benzofuran-2-yl]methyl}amine,
(±)-{[5-chloro-7-(2,6-dichlorophenyl)-2,3-dihydro-1-benzofuran-2-yl]methyl}amine,
(+)-{[5-chloro-7-(2,6-dichlorophenyl)-2,3-dihydro-1-benzofuran-2-yl]methyl}amine,
(−)-{[5-chloro-7-(2,6-dichlorophenyl)-2,3-dihydro-1-benzofuran-2-yl]methyl}amine,
(±)-{[(5-chloro-7-pyridin-3-yl-2,3-dihydro-1-benzofuran-2-yl)methyl]amine,
(±)-N-{[5-chloro-7-(2,6-dimethylphenyl)-2,3-dihydro-1-benzofuran-2-
yl]methyl}cyclopropanamine,
(±)-N-{[5-chloro-7-(2,6-dimethylphenyl)-2,3-dihydro-1-benzofuran-2-
yl]methyl}(cyclopropylmethyl)amine,
(±)-N-{[5-chloro-7-(2,6-dimethylphenyl)-2,3-dihydro-1-benzofuran-2-yl]methyl}ethanamine,
(±)-{[(5-methyl-7-phenyl-2,3-dihydro-1-benzofuran-2-yl)methyl]amine,
(±)-{[7-(2-methylphenyl)-5-methyl-2,3-dihydro-1-benzofuran-2-yl]methyl}amine,
(±)-{[7-(2-fluorophenyl)-5-methyl-2,3-dihydro-1-benzofuran-2-yl]methyl}amine,
(±)-{[7-(2-methoxyphenyl)-5-methyl-2,3-dihydro-1-benzofuran-2-yl]methyl}amine,
(±)-{[7-(2-chlorophenyl)-5-methyl-2,3-dihydro-1-benzofuran-2-yl]methyl}amine,
(±)-({5-methyl-7-[2-(trifluoromethyl)phenyl]-2,3-dihydro-1-benzofuran-2-yl}methyl)amine,
(±)-{[7-(3-chlorophenyl)-5-methyl-2,3-dihydro-1-benzofuran-2-yl]methyl}amine,
(±)-{[7-(3-methylphenyl)-5-methyl-2,3-dihydro-1-benzofuran-2-yl]methyl}amine,
(±)-{[7-(4-methylphenyl)-5-methyl-2,3-dihydro-1-benzofuran-2-yl]methyl}amine,
(±)-{[7-(4-fluorophenyl)-5-methyl-2,3-dihydro-1-benzofuran-2-yl]methyl}amine,
(±)-{[7-(4-chlorophenyl)-5-methyl-2,3-dihydro-1-benzofuran-2-yl]methyl}amine,
(±)-{[7-(4-methoxyphenyl)-5-methyl-2,3-dihydro-1-benzofuran-2-yl]methyl}amine,
(±)-{[7-(2,3-dimethoxyphenyl)-5-methyl-2,3-dihydro-1-benzofuran-2-yl]methyl}amine,
(−)-{[7-(2,3-dimethoxyphenyl)-5-methyl-2,3-dihydro-1-benzofuran-2-yl]methyl}amine,
(+)-{[7-(2,3-dimethoxyphenyl)-5-methyl-2,3-dihydro-1-benzofuran-2-yl]methyl}amine,
(±)-{[7-(2,4-dichlorophenyl)-5-methyl-2,3-dihydro-1-benzofuran-2-yl]methyl}amine,
(−)-{[7-(2,4-dichlorophenyl)-5-methyl-2,3-dihydro-1-benzofuran-2-yl]methyl}amine,
(+)-{[7-(2,4-dichlorophenyl)-5-methyl-2,3-dihydro-1-benzofuran-2-yl]methyl}amine,
(±)-{[7-(2,5-dichlorophenyl)-5-methyl-2,3-dihydro-1-benzofuran-2-yl]methyl}amine,
(±)-{[7-(2,6-dimethylphenyl)-5-methyl-2,3-dihydro-1-benzofuran-2-yl]methyl}amine,
(±)-{[7-(2,6-dichlorophenyl)-5-methyl-2,3-dihydro-1-benzofuran-2-yl]methyl}amine,
(−)-{[7-(2,6-dichlorophenyl)-5-methyl-2,3-dihydro-1-benzofuran-2-yl]methyl}amine,
(+)-{[7-(2,6-dichlorophenyl)-5-methyl-2,3-dihydro-1-benzofuran-2-yl]methyl}amine,
(±)-{[5-ethyl-7-(2-methylphenyl)-2,3-dihydro-1-benzofuran-2-yl]methyl}amine,
(±)-{[5-ethyl-7-(2-chlorophenyl)-2,3-dihydro-1-benzofuran-2-yl]methyl}amine,
(±)-{[5-(trifluoromethyl)-7-(2-fluorophenyl)-2,3-dihydro-1-benzofuran-2-yl]methyl}amine,
(±)-{[5-(trifluoromethyl)-7-(2-methylphenyl)-2,3-dihydro-1-benzofuran-2-yl]methyl}amine,
(±)-{[5-(trifluoromethyl)-7-(2-chlorophenyl)-2,3-dihydro-1-benzofuran-2-yl]methyl}amine,
(±)-{[5-(trifluoromethyl)-7-(2-methoxyphenyl)-2,3-dihydro-1-benzofuran-2-yl]methyl}amine,
(±)-{[5-(trifluoromethyl)-7-(2-(trifluoromethyl)phenyl)-2,3-dihydro-1-benzofuran-2-
yl]methyl}amine,
(±)-{[5-(trifluoromethyl)-7-(3-methylphenyl)-2,3-dihydro-1-benzofuran-2-yl]methyl}amine,
(±)-{[5-(trifluoromethyl)-7-(3-fluorophenyl)-2,3-dihydro-1-benzofuran-2-yl]methyl}amine,
(±)-{[5-(trifluoromethyl)-7-(3-chlorophenyl)-2,3-dihydro-1-benzofuran-2-yl]methyl}amine,
(±)-{[5-(trifluoromethyl)-7-(3-methoxyphenyl)-2,3-dihydro-1-benzofuran-2-yl]methyl}amine,
(±)-{[5-(trifluoromethyl)-7-(3-(trifluoromethyl)phenyl)-2,3-dihydro-1-benzofuran-2-
yl]methyl}amine,
(±)-{[5-(trifluoromethyl)-7-(4-methylphenyl)-2,3-dihydro-1-benzofuran-2-yl]methyl}amine,
(±)-{[5-(trifluoromethyl)-7-(4-fluorophenyl)-2,3-dihydro-1-benzofuran-2-yl]methyl}amine,
(±)-{[5-(trifluoromethyl)-7-(4-chlorophenyl)-2,3-dihydro-1-benzofuran-2-yl]methyl}amine,
(±)-{[5-(trifluoromethyl)-7-(4-methoxyphenyl)-2,3-dihydro-1-benzofuran-2-yl]methyl}amine,
(±)-{[5-(trifluoromethyl)-7-(4-(trifluoromethyl)phenyl)-2,3-dihydro-1-benzofuran-2-
yl]methyl}amine,
(±)-{[7-(2,3-dimethylphenyl)-5-(trifluoromethyl)-2,3-dihydro-1-benzofuran-2-yl]methyl}amine,
(±)-{[7-(2,3-difluorophenyl)-5-(trifluoromethyl)-2,3-dihydro-1-benzofuran-2-yl]methyl}amine,
(±)-{[7-(2,3-dichlorophenyl)-5-(trifluoromethyl)-2,3-dihydro-1-benzofuran-2-yl]methyl}amine,
(±)-{[7-(2,3-dimethoxyphenyl)-5-(trifluoromethyl)-2,3-dihydro-1-benzofuran-2-
yl]methyl}amine,
(±)-{[7-(2,4-difluorophenyl)-5-(trifluoromethyl)-2,3-dihydro-1-benzofuran-2-yl]methyl}amine,
(±)-{[7-(2,4-dimethoxyphenyl)-5-(trifluoromethyl)-2,3-dihydro-1-benzofuran-2-
yl]methyl}amine,
(±)-{[7-(3,4-difluorophenyl)-5-(trifluoromethyl)-2,3-dihydro-1-benzofuran-2-yl]methyl}amine,
(±)-{[7-(3-chloro-4-fluorophenyl)-5-(trifluoromethyl)-2,3-dihydro-1-benzofuran-2-
yl]methyl}amine,
(±)-{[7-(2,5-difluorophenyl)-5-(trifluoromethyl)-2,3-dihydro-1-benzofuran-2-yl]methyl}amine,
(±)-{[7-(2,5-dichlorophenyl)-5-(trifluoromethyl)-2,3-dihydro-1-benzofuran-2-yl]methyl}amine,
(±)-{[7-(2,6-dimethylphenyl)-5-(trifluoromethyl)-2,3-dihydro-1-benzofuran-2-yl]methyl}amine,
(±)-4-[2-(aminomethyl)-5-(trifluoromethyl)-2,3-dihydro-1-benzofuran-7-yl]benzonitrile
(±)-{[7-(3-furyl)-5-(trifluoromethyl)-2,3-dihydro-1-benzofuran-2-yl]methyl}amine,
(±)-{[7-thien-3-yl-5-(trifluoromethyl)-2,3-dihydro-1-benzofuran-2-yl]methyl}amine,
(±)-{[7-pyridin-3-yl-5-(trifluoromethyl)-2,3-dihydro-1-benzofuran-2-yl]methyl}amine,
(±)-{[7-(2-fluorophenyl)-5-methoxy-2,3-dihydro-1-benzofuran-2-yl]methyl}amine,
(±)-{[7-(2-chlorophenyl)-5-methoxy-2,3-dihydro-1-benzofuran-2-yl]methyl}amine,
(±)-{[7-(2-methylphenyl)-5-methoxy-2,3-dihydro-1-benzofuran-2-yl]methyl}amine,
(±)-{[7-(2-methoxyphenyl)-5-methoxy-2,3-dihydro-1-benzofuran-2-yl]methyl}amine,
(±)-{[5-methoxy-7-(3-thienyl)-2,3-dihydro-1-benzofuran-2-yl]methyl}amine,
(±)-{[7-(2,3-difluorophenyl)-5-methoxy-2,3-dihydro-1-benzofuran-2-yl]methyl}amine,
(±)-{[7-(2,3-dichlorophenyl)-5-methoxy-2,3-dihydro-1-benzofuran-2-yl]methyl}amine,
(±)-{[7-(2,3-dimethylphenyl)-5-methoxy-2,3-dihydro-1-benzofuran-2-yl]methyl}amine,
(±)-{[7-(2,4-difluorophenyl)-5-methoxy-2,3-dihydro-1-benzofuran-2-yl]methyl}amine,
(±)-{[7-(2,4-dichlorophenyl)-5-methoxy-2,3-dihydro-1-benzofuran-2-yl]methyl}amine,
(±)-{[7-(2,5-difluorophenyl)-5-methoxy-2,3-dihydro-1-benzofuran-2-yl]methyl}amine,
(±)-{[7-(2,5-dichlorophenyl)-5-methoxy-2,3-dihydro-1-benzofuran-2-yl]methyl}amine,
(+)-{[7-(2,5-dichlorophenyl)-5-methoxy-2,3-dihydro-1-benzofuran-2-yl]methyl}amine,
(−)-{[7-(2,5-dichlorophenyl)-5-methoxy-2,3-dihydro-1-benzofuran-2-yl]methyl}amine,
(±)-{[7-(2,5-dimethylphenyl)-5-methoxy-2,3-dihydro-1-benzofuran-2-yl]methyl}amine,
(±)-{[7-(2,5-dimethoxylphenyl)-5-methoxy-2,3-dihydro-1-benzofuran-2-yl]methyl}amine,
(±)-{[7-(5-chloro-2-methoxyphenyl)-5-methoxy-2,3-dihydro-1-benzofuran-2-yl]methyl}amine,
(±)-{[7-(3-chloro-4-fluorophenyl)-5-methoxy-2,3-dihydro-1-benzofuran-2-yl]methyl}amine,
(±)-{[7-(2,6-dimethylphenyl)-5-methoxy-2,3-dihydro-1-benzofuran-2-yl]methyl}amine,
(±)-[(N-methyl-1-[7-(2-methoxyphenyl)-2,3-dihydro-1-benzofuran-2-yl]methanamine,
(±)-[(N-methyl-1-[7-(3-methylphenyl)-2,3-dihydro-1-benzofuran-2-yl]methanamine,
(±)-[(N-methyl-1-[7-(3-fluorophenyl)-2,3-dihydro-1-benzofuran-2-yl]methanamine,
(±)-[(N-methyl-1-[7-(3-chlorophenyl)-2,3-dihydro-1-benzofuran-2-yl]methanamine,
(±)-[(N-methyl-1-[7-(3-methoxyphenyl)-2,3-dihydro-1-benzofuran-2-yl]methanamine,
(±)-[(N-methyl-1-[7-(4-methylphenyl)-2,3-dihydro-1-benzofuran-2-yl]methanamine,
(±)-[(N-methyl-1-[7-(4-fluorophenyl)-2,3-dihydro-1-benzofuran-2-yl]methanamine,
(±)-[(N-methyl-1-[7-(4-chlorophenyl)-2,3-dihydro-1-benzofuran-2-yl]methanamine,
(±)-[(N-methyl-1-[7-(4-methoxyphenyl)-2,3-dihydro-1-benzofuran-2-yl]methanamine,
(±)-[(N-methyl-1-[7-(2,3-dimethylphenyl)-2,3-dihydro-1-benzofuran-2-yl]methanamine,
(±)-[(N-methyl-1-[7-(2,3-dimethoxyphenyl)-2,3-dihydro-1-benzofuran-2-yl]methanamine,
(±)-[(N-methyl-1-[7-(2,4-difluorophenyl)-2,3-dihydro-1-benzofuran-2-yl]methanamine,
(±)-[(N-methyl-1-[7-(2,4-dichlorophenyl)-2,3-dihydro-1-benzofuran-2-yl]methanamine,
(±)-[(N-methyl-1-[7-(2,4-dimethoxyphenyl)-2,3-dihydro-1-benzofuran-2-yl]methanamine,
(±)-[(N-methyl-1-[7-(2,5-dimethylphenyl)-2,3-dihydro-1-benzofuran-2-yl]methanamine,
(±)-[(N-methyl-1-[7-(2,5-difluorophenyl)-2,3-dihydro-1-benzofuran-2-yl]methanamine,
(±)-[(N-methyl-1-[7-(2,5-dichlorophenyl)-2,3-dihydro-1-benzofuran-2-yl]methanamine,
(±)-[(N-methyl-1-[7-(5-chloro-2-methoxyphenyl)-2,3-dihydro-1-benzofuran-2-yl]methanamine,
(±)-[(N-methyl-1-[7-(5-chloro-2-methylphenyl)-2,3-dihydro-1-benzofuran-2-yl]methanamine,
(±)-[(N-methyl-1-[7-(2,6-dimethylphenyl)-2,3-dihydro-1-benzofuran-2-yl]methanamine,
(±)-[(N-methyl-1-[7-(2,6-dichlorophenyl)-2,3-dihydro-1-benzofuran-2-yl]methanamine,
(−)-{[7-(2,6-dichlorophenyl)-2,3-dihydro-1-benzofuran-2-yl]methyl}methylamine,
(+)-{[7-(2,6-dichlorophenyl)-2,3-dihydro-1-benzofuran-2-yl]methyl}methylamine,
(±)-N-methyl-1-(7-pyridin-3-yl-2,3-dihydro-1-benzofuran-2-yl)methanamine,
(±)-[(N-methyl-1-[7-(2,3-difluorophenyl)-2,3-dihydro-1-benzofuran-2-yl]methanamine,
(±)-{[5-fluoro-7-(2-fluorophenyl)-2,3-dihydro-1-benzofuran-2-yl]methyl}methylamine,
(±)-{[5-fluoro-7-(2-chlorophenyl)-2,3-dihydro-1-benzofuran-2-yl]methyl}methylamine,
(±)-{[5-fluoro-7-(2-methylphenyl)-2,3-dihydro-1-benzofuran-2-yl]methyl}methylamine,
(−)-{[5-fluoro-7-(2-methylphenyl)-2,3-dihydro-1-benzofuran-2-yl]methyl}methylamine,
(+)-{[5-fluoro-7-(2-methylphenyl)-2,3-dihydro-1-benzofuran-2-yl]methyl}methylamine,
(±)-{[5-fluoro-7-(2-methoxyphenyl)-2,3-dihydro-1-benzofuran-2-yl]methyl}methylamine,
(±)-{[7-(2,3-dimethoxyphenyl)-5-fluoro-2,3-dihydro-1-benzofuran-2-yl]methyl}methylamine,
(±)-[7-(2,4-dichlorophenyl)-5-fluoro-2,3-dihydro-1-benzofuran-2-yl]methyl}methylamine,
(−)-[7-(2,4-dichlorophenyl)-5-fluoro-2,3-dihydro-1-benzofuran-2-yl]methyl}methylamine,
(+)-[7-(2,4-dichlorophenyl)-5-fluoro-2,3-dihydro-1-benzofuran-2-yl]methyl}methylamine,
(±)-{[7-(2,5-difluorophenyl)-5-fluoro-2,3-dihydro-1-benzofuran-2-yl]methyl}methylamine,
(±)-{[7-(2,5-dichlorophenyl)-5-fluoro-2,3-dihydro-1-benzofuran-2-yl]methyl}methylamine,
(+)-{[5-fluoro-7-(2,5-dichlorophenyl)-2,3-dihydro-1-benzofuran-2-yl]methyl}methylamine,
(−)-{[5-fluoro-7-(2,5-dichlorophenyl)-2,3-dihydro-1-benzofuran-2-yl]methyl}methylamine,
(±)-{[7-(2,5-dimethylphenyl)-5-fluoro-2,3-dihydro-1-benzofuran-2-yl]methyl}methylamine,
(±)-{[7-(2,6-dimethylphenyl)-5-fluoro-2,3-dihydro-1-benzofuran-2-yl]methyl}methylamine,
(±)-{[7-(2,6-dichlorophenyl)-5-fluoro-2,3-dihydro-1-benzofuran-2-yl]methyl}methylamine,
(±)-{[5-fluoro-7-(5-methoxy-2-methylphenyl)-2,3-dihydro-1-benzofuran-2-
yl]methyl}methylamine,
(±)-{[5-fluoro-7-(2-methoxy-5-methylphenyl)-2,3-dihydro-1-benzofuran-2-
yl]methyl}methylamine,
(±)-{[7-(5-chloro-2-methoxyphenyl)-5-fluoro-2,3-dihydro-1-benzofuran-2-
yl]methyl}methylamine,
(±)-[(5-fluoro-7-pyridin-3-yl-2,3-dihydro-1-benzofuran-2-yl)methyl]methylamine,
(±)-[(5-chloro-7-(2-methoxyphenyl)-2,3-dihydro-1-benzofuran-2-yl]methyl}methylamine,
(±)-{[5-chloro-7-(2,6-dimethylphenyl)-2,3-dihydro-1-benzofuran-2-yl]methyl}methylamine,
(±)-[(5-chloro-7-(2-fluorophenyl)-2,3-dihydro-1-benzofuran-2-yl]methyl}methylamine,
(±)-{[5-chloro-7-(2,3-difluorophenyl)-2,3-dihydro-1-benzofuran-2-yl]methyl}methylamine,
(±)-{[5-chloro-7-(2,3-dichlorophenyl)-2,3-dihydro-1-benzofuran-2-yl]methyl}methylamine,
(±)-{[5-chloro-7-(2,3-dimethylphenyl)-2,3-dihydro-1-benzofuran-2-yl]methyl}methylamine,
(±)-{[5-chloro-7-(2,3-dimethoxyphenyl)-2,3-dihydro-1-benzofuran-2-yl]methyl}methylamine,
(±)-{[5-chloro-7-(2,4-difluorophenyl)-2,3-dihydro-1-benzofuran-2-yl]methyl}methylamine,
(±)-{[5-chloro-7-(2,4-dichlorophenyl)-2,3-dihydro-1-benzofuran-2-yl]methyl}methylamine,
(±)-{[5-chloro-7-(2,4-dimethoxyphenyl)-2,3-dihydro-1-benzofuran-2-yl]methyl}methylamine,
(±)-{[5-chloro-7-(2,5-difluorophenyl)-2,3-dihydro-1-benzofuran-2-yl]methyl}methylamine,
(±)-{[5-chloro-7-(2,5-dichlorophenyl)-2,3-dihydro-1-benzofuran-2-yl]methyl}methylamine,
(±)-{[5-chloro-7-(5-chloro-2-methoxyphenyl)-2,3-dihydro-1-benzofuran-2-
yl]methyl}methylamine,
(±)-{[5-chloro-7-(3.4-difluorophenyl)-2,3-dihydro-1-benzofuran-2-yl]methyl}methylamine,
(±)-[5-chloro-7-(3-chloro-4-fluorophenyl)-2,3-dihydro-1-benzofuran-2-yl]methyl}methylamine,
(±)-{[7-(2-fluorophenyl)-5-methyl-2,3-dihydro-1-benzofuran-2-yl]methyl}methylamine,
(±)-{[7-(2-chlorophenyl)-5-methyl-2,3-dihydro-1-benzofuran-2-yl]methyl}methylamine,
(±)-{[7-(2-methoxyphenyl)-5-methyl-2,3-dihydro-1-benzofuran-2-yl]methyl}methylamine,
(±)-{[7-(3-methylphenyl)-5-methyl-2,3-dihydro-1-benzofuran-2-yl]methyl}methylamine,
(±)-{[7-(3-chlorophenyl)-5-methyl-2,3-dihydro-1-benzofuran-2-yl]methyl}methylamine,
(±)-{[7-(4-methylphenyl)-5-methyl-2,3-dihydro-1-benzofuran-2-yl]methyl}methylamine,
(±)-{[7-(4-chlorophenyl)-5-methyl-2,3-dihydro-1-benzofuran-2-yl]methyl}methylamine,
(±)-{[7-(4-fluorophenyl)-5-methyl-2,3-dihydro-1-benzofuran-2-yl]methyl}methylamine,
(±)-{[7-(4-methoxyphenyl)-5-methyl-2,3-dihydro-1-benzofuran-2-yl]methyl}methylamine,
(±)-{[7-(2,3-dimethoxyphenyl)-5-methyl-2,3-dihydro-1-benzofuran-2-yl]methyl}methylamine,
(±)-{[7-(2,4-dichlorophenyl)-5-methyl-2,3-dihydro-1-benzofuran-2-yl]methyl}methylamine,
(±)-{[7-(2,5-dichlorophenyl)-5-methyl-2,3-dihydro-1-benzofuran-2-yl]methyl}methylamine,
(+)-{[7-(2,5-dichlorophenyl)-5-methyl-2,3-dihydro-1-benzofuran-2-yl]methyl}methylamine,
(−)-{[7-(2,5-dichlorophenyl)-5-methyl-2,3-dihydro-1-benzofuran-2-yl]methyl}methylamine,
(±)-{[7-(2,6-dimethylphenyl)-5-methyl-2,3-dihydro-1-benzofuran-2-yl]methyl}methylamine,
(±)-{[7-(2,6-dichlorophenyl)-5-methyl-2,3-dihydro-1-benzofuran-2-yl]methyl}methylamine,
(±)-{[7-(2-fluorophenyl)-5-methoxy-2,3-dihydro-1-benzofuran-2-yl]methyl}methylamine,
(±)-{[7-(2-chlorophenyl)-5-methoxy-2,3-dihydro-1-benzofuran-2-yl]methyl}methylamine,
(±)-{[7-(2-methylphenyl)-5-methoxy-2,3-dihydro-1-benzofuran-2-yl]methyl}methylamine,
(±)-{[7-(2,3-difluorophenyl)-5-methoxy-2,3-dihydro-1-benzofuran-2-yl]methyl}methylamine,
(±)-{[7-(2,3-dichlorophenyl)-5-methoxy-2,3-dihydro-1-benzofuran-2-yl]methyl}methylamine,
(±)-{[7-(2,3-dimethylphenyl)-5-methoxy-2,3-dihydro-1-benzofuran-2-yl]methyl}methylamine,
(±)-{[7-(2,4-difluorophenyl)-5-methoxy-2,3-dihydro-1-benzofuran-2-yl]methyl}methylamine,
(±)-{[7-(2,5-difluorophenyl)-5-methoxy-2,3-dihydro-1-benzofuran-2-yl]methyl}methylamine,
(±)-{[7-(2,5-dichlorophenyl)-5-methoxy-2,3-dihydro-1-benzofuran-2-yl]methyl}methylamine,
(±)-{[7-(2,5-dimethylphenyl)-5-methoxy-2,3-dihydro-1-benzofuran-2-yl]methyl}methylamine,
(±)-{[7-(2,5-dimethoxyphenyl)-5-methoxy-2,3-dihydro-1-benzofuran-2-yl]methyl}methylamine,
(±)-{[7-(5-chloro-2-methoxyphenyl)-5-methoxy-2,3-dihydro-1-benzofuran-2-
yl]methyl}methylamine,
(±)-{[7-(2,6-dimethylphenyl)-5-methoxy-2,3-dihydro-1-benzofuran-2-yl]methyl}methylamine,
(±)-N-methyl-1-[7-(2,3-difluorophenyl)-5-(trifluoromethyl)-2,3-dihydro-1-benzofuran-2-
yl]methanamine,
(±)-N-methyl-1-[7-(3,4-difluorophenyl)-5-(trifluoromethyl)-2,3-dihydro-1-benzofuran-2-
yl]methanamine,
(±)-N-methyl-1-[7-(2,5-difluorophenyl)-5-(trifluoromethyl)-2,3-dihydro-1-benzofuran-2-
yl]methanamine,
(±)-N-methyl-1-[7-(2,3-dichlorophenyl)-5-(trifluoromethyl)-2,3-dihydro-1-benzofuran-2-
yl]methanamine,
(±)-{[7-(3-chloro-4-fluorophenyl)-5-(trifluoromethyl)-2,3-dihydro-1-benzofuran-2-
yl]methyl}methylamine,
(±)-N-methyl-1-[7-(2,4-dimethoxyphenyl)-5-(trifluoromethyl)-2,3-dihydro-1-benzofuran-2-
yl]methanamine,
(+){[7-(2,6-dichlorophenyl)-5-fluoro-2,3-dihydro-1-benzofuran-2-yl]methyl}methylamine,
(−){[7-(2,6-dichlorophenyl)-5-fluoro-2,3-dihydro-1-benzofuran-2-yl]methyl}methylamine,
(R)-[7-(2-chloro-phenyl)-(5-Fluoro-2,3-dihydro-benzofuran-2-ylmethyl)methyl-amine,
(R)-[7-(2,6-dichloro-phenyl)-5-Fluoro-2,3-dihydro-benzofuran-2-ylmethyl]ethylamine,
(R)-[7-(2,6-dichloro-phenyl)-5-Fluoro-2,3-dihydro-benzofuran-2-ylmethyl]dimethylamine,
{[(2R)-7-(5-chloro-2-methylphenyl)-5-fluoro-2,3-dihydro-1-benzofuran-2-yl]methyl}amine,
{[(2R)-7-(4-chloro-2-methylphenyl)-5-fluoro-2,3-dihydro-1-benzofuran-2-yl]methyl}amine,
(−)-{[7-(2,6-dichlorophenyl)-5-fluoro-2,3-dihydro-1-benzofuran-2-yl]methyl}amine,
(+)-{[7-(2,6 dichlorophenyl)-5-fluoro-2,3-dihydro-1-benzofuran-2-yl]methyl}amine,
(±)-{2-[6-chloro-7-(2-chlorophenyl)-2,3-dihydro-1-benzofuran-2-yl]ethyl}amine,
(±)-{2-[7-(2,6-dichlorophenyl)-5-fluoro-2,3-dihydro-1-benzofuran-2-yl]ethyl}amine,
(±)-{2-[7-(2-methoxyphenyl)-5-methoxy-2,3-dihydro-1-benzofuran-2-yl]ethyl}amine,
(±)-{N-methyl-1-[(7-(2,4,6-trichlorophenyl)-2,3-dihydro-1-benzofuran-2-yl]methanamine,
(+)-{N-methyl-1-[(7-(2,4,6-trichlorophenyl)-2,3-dihydro-1-benzofuran-2-yl]methanamine,
(−)-{N-methyl-1-[(7-(2,4,6-trichlorophenyl)-2,3-dihydro-1-benzofuran-2-yl]methanamine,
(+)-{[7-(2,6-dimethylphenyl)-5-fluoro-2,3-dihydro-1-benzofuran-2-yl]methyl}methylamine,
(−)-{[7-(2,6-dimethylphenyl)-5-fluoro-2,3-dihydro-1-benzofuran-2-yl]methyl}methylamine,
(−)-{[7-(2,6-dimethylphenyl)-5-methoxy-2,3-dihydro-1-benzofuran-2-yl]methyl}methylamine,
(+)-{[7-(2,6-dimethylphenyl)-5-methoxy-2,3-dihydro-1-benzofuran-2-yl]methyl}methylamine,
(+)-{[5-chloro-7-(2,5-dichlorophenyl)-2,3-dihydro-1-benzofuran-2-yl]methyl}methylamine,
(−)-{[5-chloro-7-(2,5-dichlorophenyl)-2,3-dihydro-1-benzofuran-2-yl]methyl}methylamine,
(−)-{[5-chloro-7-(2,6-dimethylphenyl)-2,3-dihydro-1-benzofuran-2-yl]methyl}methylamine,
(+)-{[5-chloro-7-(2,6-dimethylphenyl)-2,3-dihydro-1-benzofuran-2-yl]methyl}methylamine,
(+)-{[7-(2,3-dimethoxyphenyl)-5-methyl-2,3-dihydro-1-benzofuran-2-yl]methyl}methylamine,
(−)-{[7-(2,3-dimethoxyphenyl)-5-methyl-2,3-dihydro-1-benzofuran-2-yl]methyl}methylamine,
(−)-{[7-(2,3-dimethoxyphenyl)-5-fluoro-2,3-dihydro-1-benzofuran-2-yl]methyl}methylamine, or
(+)-{[7-(2,3-dimethoxyphenyl)-5-fluoro-2,3-dihydro-1-benzofuran-2-yl]methyl}methylamine;
or a pharmaceutically acceptable salt thereof.

or a pharmaceutically acceptable salt thereof.

In certain embodiments, exemplary compounds of formula I are as set forth in Table 1-a, below.

TABLE 1-a
Exemplary Compounds of formula I:

(±)-1-(7-phenyl-2,3-dihydro-1-benzofuran-2-yl)methanamine,
(+)-(1-(7-phenyl-2,3-dihydro-1-benzofuran-2-yl)methanamine,
(−)-1-(7-phenyl-2,3-dihydro-1-benzofuran-2-yl)methanamine,
(±)-1-[7-(2-methylphenyl)-2,3-dihydro-1-benzofuran-2-yl]methanamine,
(+)-1-[7-(2-methylphenyl)-2,3-dihydro-1-benzofuran-2-yl]methanamine,
(−)-1-[7-(2-methylphenyl)-2,3-dihydro-1-benzofuran-2-yl]methanamine,
(±)-1-[7-(2-fluorophenyl)-2,3-dihydro-1-benzofuran-2-yl]methanamine,
(+)-1-[7-(2-fluorophenyl)-2,3-dihydro-1-benzofuran-2-yl]methanamine,
(−)-1-[7-(2-fluorophenyl)-2,3-dihydro-1-benzofuran-2-yl]methanamine,
(±)-1-{7-[2-(trifluoromethyl)phenyl]-2,3-dihydro-1-benzofuran-2-yl}methanamine,
(−)-1-{7-[2-(trifluoromethyl)phenyl]-2,3-dihydro-1-benzofuran-2-yl}methanamine,
(+)-1-{7-[2-(trifluoromethyl)phenyl]-2,3-dihydro-1-benzofuran-2-yl}methanamine,
(±)-1-[7-(2,6-dimethylphenyl)-2,3-dihydro-1-benzofuran-2-yl]methanamine,
(−)-1-[7-(2,6-dimethylphenyl)-2,3-dihydro-1-benzofuran-2-yl]methanamine,
(+)-1-[7-(2,6-dimethylphenyl)-2,3-dihydro-1-benzofuran-2-yl]methanamine,
(±)-1-[7-(2-methoxyphenyl)-2,3-dihydro-1-benzofuran-2-yl]methanamine,
(±)-1-[7-(2-chlorophenyl)-2,3-dihydro-1-benzofuran-2-yl]methanamine,
(±)-1-[7-(2,4-dichlorophenyl)-2,3-dihydro-1-benzofuran-2-yl]methanamine,
(±)-1-[5-chloro-7-(2-chlorophenyl)-2,3-dihydro-1-benzofuran-2-yl]methanamine,
(±)-1-[5-chloro-7-(2-methylphenyl)-2,3-dihydro-1-benzofuran-2-yl]methanamine,
(+)-1-[5-chloro-7-(2-methylphenyl)-2,3-dihydro-1-benzofuran-2-yl]methanamine,
(−)-1-[5-chloro-7-(2-methylphenyl)-2,3-dihydro-1-benzofuran-2-yl]methanamine,
(±)-1-[4-fluoro-7-(2-methylphenyl)-2,3-dihydro-1-benzofuran-2-yl]methanamine,
(±)-1-[7-(2-chlorophenyl)-5-fluoro-2,3-dihydro-1-benzofuran-2-yl]methanamine,
(+)-1-[7-(2-chlorophenyl)-5-fluoro-2,3-dihydro-1-benzofuran-2-yl]methanamine,
(−)-1-[7-(2-chlorophenyl)-5-fluoro-2,3-dihydro-1-benzofuran-2-yl]methanamine,
(±)-1-[5-fluoro-7-(2-methylphenyl)-2,3-dihydro-1-benzofuran-2-yl]methanamine,
(+)-1-[5-fluoro-7-(2-methylphenyl)-2,3-dihydro-1-benzofuran-2-yl]methanamine,
(−)-1-[5-fluoro-7-(2-methylphenyl)-2,3-dihydro-1-benzofuran-2-yl]methanamine,
(±)-1-[5-fluoro-7-(2-fluorophenyl)-2,3-dihydro-1-benzofuran-2-yl]methanamine,
(±)-1-{5-fluoro-7-[2-(trifluoromethyl)phenyl]-2,3-dihydro-1-benzofuran-2-yl}methanamine,
(±)-1-[4,5-difluoro-7-(2-methylphenyl)-2,3-dihydro-1-benzofuran-2-yl]methanamine,
(±)-1-(5-chloro-2-methyl-7-phenyl-2,3-dihydro-1-benzofuran-2-yl)methanamine,
(+)-1-[7-(2-methoxyphenyl)-2,3-dihydro-1-benzofuran-2-yl]methanamine,
(−)-1-[7-(2-chlorophenyl)-2,3-dihydro-1-benzofuran-2-yl]methanamine,
(+)-1-[7-(2-chlorophenyl)-2,3-dihydro-1-benzofuran-2-yl]methanamine,
(±)-1-[7-(2,6-difluorophenyl)-2,3-dihydro-1-benzofuran-2-yl]methanamine,
(±)-1-[7-(2,6-dichlorophenyl)-2,3-dihydro-1-benzofuran-2-yl]methanamine,
(−)-1-[7-(2,6-dichlorophenyl)-2,3-dihydro-1-benzofuran-2-yl]methanamine,
(+)-1-[7-(2,6-dichlorophenyl)-2,3-dihydro-1-benzofuran-2-yl]methanamine,
(±)-1-[7-(2,4-dimethoxyphenyl)-2,3-dihydro-1-benzofuran-2-yl]methanamine,
(−)-1-[7-(2,4-dimethoxyphenyl)-2,3-dihydro-1-benzofuran-2-yl]methanamine,
(+)-1-[7-(2,4-dimethoxyphenyl)-2,3-dihydro-1-benzofuran-2-yl]methanamine,
(±)-1-[7-(2,4-difluorophenyl)-2,3-dihydro-1-benzofuran-2-yl]methanamine,
(+)-1-[7-(2,4-difluorophenyl)-2,3-dihydro-1-benzofuran-2-yl]methanamine,
(−)-1-[7-(2,4-difluorophenyl)-2,3-dihydro-1-benzofuran-2-yl]methanamine,
(−)-1-[7-(2,4-dichlorophenyl)-2,3-dihydro-1-benzofuran-2-yl]methanamine,
(+)-1-[7-(2,4-dichlorophenyl)-2,3-dihydro-1-benzofuran-2-yl]methanamine,
(−)-1-{5-fluoro-7-[2-(trifluoromethyl)phenyl]-2,3-dihydro-1-benzofuran-2-yl}methanamine,
(+)-1-{5-fluoro-7-[2-(trifluoromethyl)phenyl]-2,3-dihydro-1-benzofuran-2-yl}methanamine,
(±)-1-[7-(2,3-dimethylphenyl)-2,3-dihydro-1-benzofuran-2-yl]methanamine,
(±)-{[7-(2,3-dimethoxyphenyl)-2,3-dihydro-1-benzofuran-2-yl]methyl}amine,
(−)-{[(7-(2,3-dimethoxyphenyl)-2,3-dihydro-1-benzofuran-2-yl]methyl}amine,
(+)-{[(7-(2,3-dimethoxyphenyl)-2,3-dihydro-1-benzofuran-2-yl]methyl}amine,
(+)-{[7-(4-chloro-2-methylphenyl)-2,3-dihydro-1-benzofuran-2-yl]methyl}amine,
(−)-{[7-(4-chloro-2-methylphenyl)-2,3-dihydro-1-benzofuran-2-yl]methyl}amine,
(±)-{[7-(2,3-difluorophenyl)-2,3-dihydro-1-benzofuran-2-yl]methyl}amine,
(±)-{[7-(2,5-dimethylphenyl)-2,3-dihydro-1-benzofuran-2-yl]methyl}amine,
(±)-{[7-(2,5-dimethoxyphenyl)-2,3-dihydro-1-benzofuran-2-yl]methyl}amine,
(±)-{[7-(2,5-dichlorophenyl)-2,3-dihydro-1-benzofuran-2-yl]methyl}amine,
(+)-{[7-(2,5-dichlorophenyl)-2,3-dihydro-1-benzofuran-2-yl]methyl}amine,
(−)-{[7-(2,5-dichlorophenyl)-2,3-dihydro-1-benzofuran--yl]methyl}amine,
(±)-{[7-(2,4,6-trichlorophenyl)-2,3-dihydro-1-benzofuran-2-yl]methyl}amine,
(±)-{[7-(4-chloro-2-methylphenyl)-2,3-dihydro-1-benzofuran-2-yl]methyl}amine,
(±)-{[7-(5-chloro-2-methylphenyl)-2,3-dihydro-1-benzofuran-2-yl]methyl}amine,
(±)-{[7-(5-chloro-2-methoxyphenyl)-2,3-dihydro-1-benzofuran-2-yl]methyl}amine,
(±)-{[5-fluoro-7-(2-methoxyphenyl)-2,3-dihydro-1-benzofuran-2-yl]methyl}amine,
(±)-{[7-(2,3-dichlorophenyl)-5-fluoro-2,3-dihydro-1-benzofuran-2-yl]methyl}amine,
(±)-{[7-(2,3-dimethoxyphenyl)-5-fluoro-2,3-dihydro-1-benzofuran-2-yl]methyl}amine,
(−)-{[7-(2,3-dimethoxyphenyl)-5-fluoro-2,3-dihydro-1-benzofuran-2-yl]methyl}amine,
(+)-{[7-(2,3-dimethoxyphenyl)-5-fluoro-2,3-dihydro-1-benzofuran-2-yl]methyl}amine,
(±)-{[7-(2,4-difluorophenyl)-5-fluoro-2,3-dihydro-1-benzofuran-2-yl]methyl}amine,
(±)-{[7-(2,4-dichlorophenyl)-5-fluoro-2,3-dihydro-1-benzofuran-2-yl]methyl}amine,
(−)-{[5-fluoro-7-(2,4-dichlorophenyl)-2,3-dihydro-1-benzofuran-2-yl]methyl}amine,
(+)-{[5-fluoro-7-(2,4-dichlorophenyl)-2,3-dihydro-1-benzofuran-2-yl]methyl}amine,
(±)-{[7-(2,4-dimethoxyphenyl)-5-fluoro-2,3-dihydro-1-benzofuran-2-yl]methyl}amine,
(±)-{[7-(2,5-difluorophenyl)-5-fluoro-2,3-dihydro-1-benzofuran-2-yl]methyl}amine,
(±)-{[7-(2,5-dichlorophenyl)-5-fluoro-2,3-dihydro-1-benzofuran-2-yl]methyl}amine,
(±)-{[7-(2,5-dimethylphenyl)-5-fluoro-2,3-dihydro-1-benzofuran-2-yl]methyl}amine,
(±)-{[7-(2,5-dimethoxyphenyl)-5-fluoro-2,3-dihydro-1-benzofuran-2-yl]methyl}amine,
(±)-{[5-fluoro-7-(5-methoxy-2-methylphenyl)-2,3-dihydro-1-benzofuran-2-yl]methyl}amine,
(±)-{[5-fluoro-7-(2-methoxy-5-methylphenyl)-2,3-dihydro-1-benzofuran-2-yl]methyl}amine,
(±)-{[7-(2,6-difluorophenyl)-5-fluoro-2,3-dihydro-1-benzofuran-2-yl]methyl}amine,
(±)-{[7-(2,6-dimethylphenyl)-5-fluoro-2,3-dihydro-1-benzofuran-2-yl]methyl}amine,
(+)-{[7-(2,6-dimethylphenyl)-5-fluoro-2,3-dihydro-1-benzofuran-2-yl]methyl}amine,
(−)-{[7-(2,6-dimethylphenyl)-5-fluoro-2,3-dihydro-1-benzofuran-2-yl]methyl}amine,
(±)-{[7-(2,6-dichlorophenyl)-5-fluoro-2,3-dihydro-1-benzofuran-2-yl]methyl}amine,
(±)-N-{[7-(2,6-dichlorophenyl)-5-fluoro-2,3-dihydro-1-benzofuran-2-
yl]methyl}cyclopropanamine,
(±)-1-cyclopropyl-N-{[7-(2,6-dichlorophenyl)-5-fluoro-2,3-dihydro-1-benzofuran-2-
yl]methyl}methanamine,
(±)-N-{[7-(2,6-dichlorophenyl)-5-fluoro-2,3-dihydro-1-benzofuran-2-yl]methyl}ethanamine,
(±)-{[7-(2,6-dichlorophenyl)-5-fluoro-2,3-dihydro-1-benzofuran-2-yl]methyl}dimethylamine,
(±)-{[5-chloro-7-(2-fluorophenyl)-2,3-dihydro-1-benzofuran-2-yl]methyl}amine,
(±)-{[5-chloro-7-(2-methoxyphenyl)-2,3-dihydro-1-benzofuran-2-yl]methyl}amine,
(±)-{[5-chloro-7-(2,3-difluorophenyl)-2,3-dihydro-1-benzofuran-2-yl]methyl}amine,
(−)-{[5-chloro-7-(2,3-difluorophenyl)-2,3-dihydro-1-benzofuran-2-yl]methyl}amine,
(+)-{[5-chloro-7-(2,3-difluorophenyl)-2,3-dihydro-1-benzofuran-2-yl]methyl}amine,
(±)-{[5-chloro-7-(2,3-dichlorophenyl)-2,3-dihydro-1-benzofuran-2-yl]methyl}amine,
(±)-{[5-chloro-7-(2,3-dimethylphenyl)-2,3-dihydro-1-benzofuran-2-yl]methyl}amine,
(±)-{[5-chloro-7-(2,3-dimethoxyphenyl)-2,3-dihydro-1-benzofuran-2-yl]methyl}amine,
(±)-{[5-chloro-7-(2,4-difluorophenyl)-2,3-dihydro-1-benzofuran-2-yl]methyl}amine,
(±)-{[5-chloro-7-(2,4-dichlorophenyl)-2,3-dihydro-1-benzofuran-2-yl]methyl}amine,
(−)-{[5-chloro-7-(2,4-dichlorophenyl)-2,3-dihydro-1-benzofuran-2-yl]methyl}amine,
(+)-{[5-chloro-7-(2,4-dichlorophenyl)-2,3-dihydro-1-benzofuran-2-yl]methyl}amine,
(±)-{[5-chloro-7-(2,4-dimethoxyphenyl)-2,3-dihydro-1-benzofuran-2-yl]methyl}amine,
(±)-{[5-chloro-7-(2,5-difluorophenyl)-2,3-dihydro-1-benzofuran-2-yl]methyl}amine,
(±)-{[5-chloro-7-(2,5-dichlorophenyl)-2,3-dihydro-1-benzofuran-2-yl]methyl}amine,
(±)-{[5-chloro-7-(5-chloro-2-methoxyphenyl)-2,3-dihydro-1-benzofuran-2-yl]methyl}amine,
(±)-{[5-chloro-7-(2,6-dimethylphenyl)-2,3-dihydro-1-benzofuran-2-yl]methyl}amine,
(−)-{[5-chloro-7-(2,6-dimethylphenyl)-2,3-dihydro-1-benzofuran-2-yl]methyl}amine,
(+)-{[5-chloro-7-(2,6-dimethylphenyl)-2,3-dihydro-1-benzofuran-2-yl]methyl}amine,
(±)-{[5-chloro-7-(2,6-dichlorophenyl)-2,3-dihydro-1-benzofuran-2-yl]methyl}amine,
(+)-{[5-chloro-7-(2,6-dichlorophenyl)-2,3-dihydro-1-benzofuran-2-yl]methyl}amine,
(−)-{[5-chloro-7-(2,6-dichlorophenyl)-2,3-dihydro-1-benzofuran-2-yl]methyl}amine,
(±)-N-{[5-chloro-7-(2,6-dimethylphenyl)-2,3-dihydro-1-benzofuran-2-
yl]methyl}cyclopropanamine,
(±)-N-{[5-chloro-7-(2,6-dimethylphenyl)-2,3-dihydro-1-benzofuran-2-
yl]methyl}(cyclopropylmethyl)amine,
(±)-N-{[5-chloro-7-(2,6-dimethylphenyl)-2,3-dihydro-1-benzofuran-2-yl]methyl}ethanamine,
(±)-{[7-(2-methylphenyl)-5-methyl-2,3-dihydro-1-benzofuran-2-yl]methyl}amine,
(±)-{[7-(2-fluorophenyl)-5-methyl-2,3-dihydro-1-benzofuran-2-yl]methyl}amine,
(±)-{[7-(2-methoxyphenyl)-5-methyl-2,3-dihydro-1-benzofuran-2-yl]methyl}amine,
(±)-{[7-(2-chlorophenyl)-5-methyl-2,3-dihydro-1-benzofuran-2-yl]methyl}amine,
(±)-({5-methyl-7-[2-(trifluoromethyl)phenyl]-2,3-dihydro-1-benzofuran-2-yl}methyl)amine,
(±)-{[7-(2,3-dimethoxyphenyl)-5-methyl-2,3-dihydro-1-benzofuran-2-yl]methyl}amine,
(−)-{[7-(2,3-dimethoxyphenyl)-5-methyl-2,3-dihydro-1-benzofuran-2-yl]methyl}amine,
(+)-{[7-(2,3-dimethoxyphenyl)-5-methyl-2,3-dihydro-1-benzofuran-2-yl]methyl}amine,
(±)-{[7-(2,4-dichlorophenyl)-5-methyl-2,3-dihydro-1-benzofuran-2-yl]methyl}amine,
(−)-{[7-(2,4-dichlorophenyl)-5-methyl-2,3-dihydro-1-benzofuran-2-yl]methyl}amine,
(+)-{[7-(2,4-dichlorophenyl)-5-methyl-2,3-dihydro-1-benzofuran-2-yl]methyl}amine,
(±)-{[7-(2,5-dichlorophenyl)-5-methyl-2,3-dihydro-1-benzofuran-2-yl]methyl}amine,
(±)-{[7-(2,6-dimethylphenyl)-5-methyl-2,3-dihydro-1-benzofuran-2-yl]methyl}amine,
(±)-{[7-(2,6-dichlorophenyl)-5-methyl-2,3-dihydro-1-benzofuran-2-yl]methyl}amine,
(−)-{[7-(2,6-dichlorophenyl)-5-methyl-2,3-dihydro-1-benzofuran-2-yl]methyl}amine,
(+)-{[7-(2,6-dichlorophenyl)-5-methyl-2,3-dihydro-1-benzofuran-2-yl]methyl}amine,
(±)-{[5-ethyl-7-(2-methylphenyl)-2,3-dihydro-1-benzofuran-2-yl]methyl}amine,
(±)-{[5-ethyl-7-(2-chlorophenyl)-2,3-dihydro-1-benzofuran-2-yl]methyl}amine,
(±)-{[5-(trifluoromethyl)-7-(2-fluorophenyl)-2,3-dihydro-1-benzofuran-2-yl]methyl}amine,
(±)-{[5-(trifluoromethyl)-7-(2-methylphenyl)-2,3-dihydro-1-benzofuran-2-yl]methyl}amine,
(±)-{[5-(trifluoromethyl)-7-(2-chlorophenyl)-2,3-dihydro-1-benzofuran-2-yl]methyl}amine,
(±)-{[5-(trifluoromethyl)-7-(2-methoxyphenyl)-2,3-dihydro-1-benzofuran-2-yl]methyl}amine,
(±)-{[5-(trifluoromethyl)-7-(2-(trifluoromethyl)phenyl)-2,3-dihydro-1-benzofuran-2-
yl]methyl}amine,
(±)-{[7-(2,3-dimethylphenyl)-5-(trifluoromethyl)-2,3-dihydro-1-benzofuran-2-yl]methyl}amine,
(±)-{[7-(2,3-difluorophenyl)-5-(trifluoromethyl)-2,3-dihydro-1-benzofuran-2-yl]methyl}amine,
(±)-{[7-(2,3-dichlorophenyl)-5-(trifluoromethyl)-2,3-dihydro-1-benzofuran-2-yl]methyl}amine,
(±)-{[7-(2,3-dimethoxyphenyl)-5-(trifluoromethyl)-2,3-dihydro-1-benzofuran-2-
yl]methyl}amine,
(±)-{[7-(2,4-difluorophenyl)-5-(trifluoromethyl)-2,3-dihydro-1-benzofuran-2-yl]methyl}amine,
(±)-{[7-(2,4-dimethoxyphenyl)-5-(trifluoromethyl)-2,3-dihydro-1-benzofuran-2-
yl]methyl}amine,
(±)-{[7-(2,5-difluorophenyl)-5-(trifluoromethyl)-2,3-dihydro-1-benzofuran-2-yl]methyl}amine,
(±)-{[7-(2,5-dichlorophenyl)-5-(trifluoromethyl)-2,3-dihydro-1-benzofuran-2-yl]methyl}amine,
(±)-{[7-(2,6-dimethylphenyl)-5-(trifluoromethyl)-2,3-dihydro-1-benzofuran-2-yl]methyl}amine,
(±)-{[7-(2-fluorophenyl)-5-methoxy-2,3-dihydro-1-benzofuran-2-yl]methyl}amine,
(±)-{[7-(2-chlorophenyl)-5-methoxy-2,3-dihydro-1-benzofuran-2-yl]methyl}amine,
(±)-{[7-(2-methylphenyl)-5-methoxy-2,3-dihydro-1-benzofuran-2-yl]methyl}amine,
(±)-{[7-(2-methoxyphenyl)-5-methoxy-2,3-dihydro-1-benzofuran-2-yl]methyl}amine,
(±)-{[7-(2,3-difluorophenyl)-5-methoxy-2,3-dihydro-1-benzofuran-2-yl]methyl}amine,
(±)-{[7-(2,3-dichlorophenyl)-5-methoxy-2,3-dihydro-1-benzofuran-2-yl]methyl}amine,
(±)-{[7-(2,3-dimethylphenyl)-5-methoxy-2,3-dihydro-1-benzofuran-2-yl]methyl}amine,
(±)-{[7-(2,4-difluorophenyl)-5-methoxy-2,3-dihydro-1-benzofuran-2-yl]methyl}amine,
(±)-{[7-(2,4-dichlorophenyl)-5-methoxy-2,3-dihydro-1-benzofuran-2-yl]methyl}amine,
(±)-{[7-(2,5-difluorophenyl)-5-methoxy-2,3-dihydro-1-benzofuran-2-yl]methyl}amine,
(±)-{[7-(2,5-dichlorophenyl)-5-methoxy-2,3-dihydro-1-benzofuran-2-yl]methyl}amine,
(+)-{[7-(2,5-dichlorophenyl)-5-methoxy-2,3-dihydro-1-benzofuran-2-yl]methyl}amine,
(−)-{[7-(2,5-dichlorophenyl)-5-methoxy-2,3-dihydro-1-benzofuran-2-yl]methyl}amine,
(±)-{[7-(2,5-dimethylphenyl)-5-methoxy-2,3-dihydro-1-benzofuran-2-yl]methyl}amine,
(±)-{[7-(2,5-dimethoxylphenyl)-5-methoxy-2,3-dihydro-1-benzofuran-2-yl]methyl}amine,
(±)-{[7-(5-chloro-2-methoxyphenyl)-5-methoxy-2,3-dihydro-1-benzofuran-2-yl]methyl}amine,
(±)-{[7-(2,6-dimethylphenyl)-5-methoxy-2,3-dihydro-1-benzofuran-2-yl]methyl}amine,
(±)-[(N-methyl-1-[7-(2-methoxyphenyl)-2,3-dihydro-1-benzofuran-2-yl]methanamine,
(±)-[(N-methyl-1-[7-(2,3-dimethylphenyl)-2,3-dihydro-1-benzofuran-2-yl]methanamine,
(±)-[(N-methyl-1-[7-(2,3-dimethoxyphenyl)-2,3-dihydro-1-benzofuran-2-yl]methanamine,
(±)-[(N-methyl-1-[7-(2,4-difluorophenyl)-2,3-dihydro-1-benzofuran-2-yl]methanamine,
(±)-[(N-methyl-1-[7-(2,4-dichlorophenyl)-2,3-dihydro-1-benzofuran-2-yl]methanamine,
(±)-[(N-methyl-1-[7-(2,4-dimethoxyphenyl)-2,3-dihydro-1-benzofuran-2-yl]methanamine,
(±)-[(N-methyl-1-[7-(2,5-dimethylphenyl)-2,3-dihydro-1-benzofuran-2-yl]methanamine,
(±)-[(N-methyl-1-[7-(2,5-difluorophenyl)-2,3-dihydro-1-benzofuran-2-yl]methanamine,
(±)-[(N-methyl-1-[7-(2,5-dichlorophenyl)-2,3-dihydro-1-benzofuran-2-yl]methanamine,
(±)-[(N-methyl-1-[7-(2,6-dimethylphenyl)-2,3-dihydro-1-benzofuran-2-yl]methanamine,
(±)-[(N-methyl-1-[7-(2,6-dichlorophenyl)-2,3-dihydro-1-benzofuran-2-yl]methanamine,
(−)-{[7-(2,6-dichlorophenyl)-2,3-dihydro-1-benzofuran-2-yl]methyl}methylamine,
(+)-{[7-(2,6-dichlorophenyl)-2,3-dihydro-1-benzofuran-2-yl]methyl}methylamine,
(±)-[(N-methyl-1-[7-(2,3-difluorophenyl)-2,3-dihydro-1-benzofuran-2-yl]methanamine,
(±)-{[5-fluoro-7-(2-fluorophenyl)-2,3-dihydro-1-benzofuran-2-yl]methyl}methylamine,
(±)-{[5-fluoro-7-(2-chlorophenyl)-2,3-dihydro-1-benzofuran-2-yl]methyl}methylamine,
(±)-{[5-fluoro-7-(2-methylphenyl)-2,3-dihydro-1-benzofuran-2-yl]methyl}methylamine,
(−)-{[5-fluoro-7-(2-methylphenyl)-2,3-dihydro-1-benzofuran-2-yl]methyl}methylamine,
(+)-{[5-fluoro-7-(2-methylphenyl)-2,3-dihydro-1-benzofuran-2-yl]methyl}methylamine,
(±)-{[5-fluoro-7-(2-methoxyphenyl)-2,3-dihydro-1-benzofuran-2-yl]methyl}methylamine,
(±)-{[7-(2,3-dimethoxyphenyl)-5-fluoro-2,3-dihydro-1-benzofuran-2-yl]methyl}methylamine,
(±)-[7-(2,4-dichlorophenyl)-5-fluoro-2,3-dihydro-1-benzofuran-2-yl]methyl}methylamine,
(−)-[7-(2,4-dichlorophenyl)-5-fluoro-2,3-dihydro-1-benzofuran-2-yl]methyl}methylamine,
(+)-[7-(2,4-dichlorophenyl)-5-fluoro-2,3-dihydro-1-benzofuran-2-yl]methyl}methylamine,
(±)-{[7-(2,5-difluorophenyl)-5-fluoro-2,3-dihydro-1-benzofuran-2-yl]methyl}methylamine,
(±)-{[7-(2,5-dichlorophenyl)-5-fluoro-2,3-dihydro-1-benzofuran-2-yl]methyl}methylamine,
(+)-{[5-fluoro-7-(2,5-dichlorophenyl)-2,3-dihydro-1-benzofuran-2-yl]methyl}methylamine,
(−)-{[5-fluoro-7-(2,5-dichlorophenyl)-2,3-dihydro-1-benzofuran-2-yl]methyl}methylamine,
(±)-{[7-(2,5-dimethylphenyl)-5-fluoro-2,3-dihydro-1-benzofuran-2-yl]methyl}methylamine,
(±)-{[7-(2,6-dimethylphenyl)-5-fluoro-2,3-dihydro-1-benzofuran-2-yl]methyl}methylamine,
(±)-{[7-(2,6-dichlorophenyl)-5-fluoro-2,3-dihydro-1-benzofuran-2-yl]methyl}methylamine,
(±)-{[5-fluoro-7-(5-methoxy-2-methylphenyl)-2,3-dihydro-1-benzofuran-2-
yl]methyl}methylamine,
(±)-{[5-fluoro-7-(2-methoxy-5-methylphenyl)-2,3-dihydro-1-benzofuran-2-
yl]methyl}methylamine,
(±)-{[7-(5-chloro-2-methoxyphenyl)-5-fluoro-2,3-dihydro-1-benzofuran-2-
yl]methyl}methylamine,
(±)-[(5-chloro-7-(2-methoxyphenyl)-2,3-dihydro-1-benzofuran-2-yl]methyl}methylamine,
(±)-{[5-chloro-7-(2,6-dimethylphenyl)-2,3-dihydro-1-benzofuran-2-yl]methyl}methylamine,
(±)-[(5-chloro-7-(2-fluorophenyl)-2,3-dihydro-1-benzofuran-2-yl]methyl}methylamine,
(±)-{[5-chloro-7-(2,3-difluorophenyl)-2,3-dihydro-1-benzofuran-2-yl]methyl}methylamine,
(±)-{[5-chloro-7-(2,3-dichlorophenyl)-2,3-dihydro-1-benzofuran-2-yl]methyl}methylamine,
(±)-{[5-chloro-7-(2,3-dimethylphenyl)-2,3-dihydro-1-benzofuran-2-yl]methyl}methylamine,
(±)-{[5-chloro-7-(2,3-dimethoxyphenyl)-2,3-dihydro-1-benzofuran-2-yl]methyl}methylamine,
(±)-{[5-chloro-7-(2,4-difluorophenyl)-2,3-dihydro-1-benzofuran-2-yl]methyl}methylamine,
(±)-{[5-chloro-7-(2,4-dichlorophenyl)-2,3-dihydro-1-benzofuran-2-yl]methyl}methylamine,
(±)-{[5-chloro-7-(2,4-dimethoxyphenyl)-2,3-dihydro-1-benzofuran-2-yl]methyl}methylamine,
(±)-{[5-chloro-7-(2,5-difluorophenyl)-2,3-dihydro-1-benzofuran-2-yl]methyl}methylamine,
(±)-{[5-chloro-7-(2,5-dichlorophenyl)-2,3-dihydro-1-benzofuran-2-yl]methyl}methylamine,
(±)-{[5-chloro-7-(5-chloro-2-methoxyphenyl)-2,3-dihydro-1-benzofuran-2-
yl]methyl}methylamine,
(±)-{[7-(2-fluorophenyl)-5-methyl-2,3-dihydro-1-benzofuran-2-yl]methyl}methylamine,
(±)-{[7-(2-chlorophenyl)-5-methyl-2,3-dihydro-1-benzofuran-2-yl]methyl}methylamine,
(±)-{[7-(2-methoxyphenyl)-5-methyl-2,3-dihydro-1-benzofuran-2-yl]methyl}methylamine,
(±)-{[7-(2,3-dimethoxyphenyl)-5-methyl-2,3-dihydro-1-benzofuran-2-yl]methyl}methylamine,
(±)-{[7-(2,4-dichlorophenyl)-5-methyl-2,3-dihydro-1-benzofuran-2-yl]methyl}methylamine,
(±)-{[7-(2,5-dichlorophenyl)-5-methyl-2,3-dihydro-1-benzofuran-2-yl]methyl}methylamine,
(+)-{[7-(2,5-dichlorophenyl)-5-methyl-2,3-dihydro-1-benzofuran-2-yl]methyl}methylamine,
(−)-{[7-(2,5-dichlorophenyl)-5-methyl-2,3-dihydro-1-benzofuran-2-yl]methyl}methylamine,
(±)-{[7-(2,6-dimethylphenyl)-5-methyl-2,3-dihydro-1-benzofuran-2-yl]methyl}methylamine,
(±)-{[7-(2,6-dichlorophenyl)-5-methyl-2,3-dihydro-1-benzofuran-2-yl]methyl}methylamine,
(±)-{[7-(2-fluorophenyl)-5-methoxy-2,3-dihydro-1-benzofuran-2-yl]methyl}methylamine,
(±)-{[7-(2-chlorophenyl)-5-methoxy-2,3-dihydro-1-benzofuran-2-yl]methyl}methylamine,
(±)-{[7-(2-methylphenyl)-5-methoxy-2,3-dihydro-1-benzofuran-2-yl]methyl}methylamine,
(±)-{[7-(2,3-difluorophenyl)-5-methoxy-2,3-dihydro-1-benzofuran-2-yl]methyl}methylamine,
(±)-{[7-(2,3-dichlorophenyl)-5-methoxy-2,3-dihydro-1-benzofuran-2-yl]methyl}methylamine,
(±)-{[7-(2,3-dimethylphenyl)-5-methoxy-2,3-dihydro-1-benzofuran-2-yl]methyl}methylamine,
(±)-{[7-(2,4-difluorophenyl)-5-methoxy-2,3-dihydro-1-benzofuran-2-yl]methyl}methylamine,
(±)-{[7-(2,5-difluorophenyl)-5-methoxy-2,3-dihydro-1-benzofuran-2-yl]methyl}methylamine,
(±)-{[7-(2,5-dichlorophenyl)-5-methoxy-2,3-dihydro-1-benzofuran-2-yl]methyl}methylamine,
(±)-{[7-(2,5-dimethylphenyl)-5-methoxy-2,3-dihydro-1-benzofuran-2-yl]methyl}methylamine,
(±)-{[7-(2,5-dimethoxyphenyl)-5-methoxy-2,3-dihydro-1-benzofuran-2-yl]methyl}methylamine,
(±)-{[7-(5-chloro-2-methoxyphenyl)-5-methoxy-2,3-dihydro-1-benzofuran-2-
yl]methyl}methylamine,
(±)-{[7-(2,6-dimethylphenyl)-5-methoxy-2,3-dihydro-1-benzofuran-2-yl]methyl}methylamine,
(±)-N-methyl-1-[7-(2,3-difluorophenyl)-5-(trifluoromethyl)-2,3-dihydro-1-benzofuran-2-
yl]methanamine,
(±)-N-methyl-1-[7-(2,3-dichlorophenyl)-5-(trifluoromethyl)-2,3-dihydro-1-benzofuran-2-
yl]methanamine,
(±)-N-methyl-1-[7-(2,4-dimethoxyphenyl)-5-(trifluoromethyl)-2,3-dihydro-1-benzofuran-2-
yl]methanamine,
(+){[7-(2,6-dichlorophenyl)-5-fluoro-2,3-dihydro-1-benzofuran-2-yl]methyl}methylamine,
(−){[7-(2,6-dichlorophenyl)-5-fluoro-2,3-dihydro-1-benzofuran-2-yl]methyl}methylamine,
(R)-[7-(2-chloro-phenyl)-(5-Fluoro-2,3-dihydro-benzofuran-2-ylmethyl)methyl-amine,
(R)-[7-(2,6-dichloro-phenyl)-5-Fluoro-2,3-dihydro-benzofuran-2-ylmethyl]ethylamine,
(R)-[7-(2,6-dichloro-phenyl)-5-Fluoro-2,3-dihydro-benzofuran-2-ylmethyl]dimethylamine,
{[(2R)-7-(5-chloro-2-methylphenyl)-5-fluoro-2,3-dihydro-1-benzofuran-2-yl]methyl}amine,
{[(2R)-7-(4-chloro-2-methylphenyl)-5-fluoro-2,3-dihydro-1-benzofuran-2-yl]methyl}amine,
(−)-{[7-(2,6-dichlorophenyl)-5-fluoro-2,3-dihydro-1-benzofuran-2-yl]methyl}amine,
(+)-{[7-(2,6 dichlorophenyl)-5-fluoro-2,3-dihydro-1-benzofuran-2-yl]methyl}amine,
(±)-{2-[6-chloro-7-(2-chlorophenyl)-2,3-dihydro-1-benzofuran-2-yl]ethyl}amine,
(±)-{2-[7-(2,6-dichlorophenyl)-5-fluoro-2,3-dihydro-1-benzofuran-2-yl]ethyl}amine,
(±)-{2-[7-(2-methoxyphenyl)-5-methoxy-2,3-dihydro-1-benzofuran-2-yl]ethyl}amine,
(±)-{N-methyl-1-[(7-(2,4,6-trichlorophenyl)-2,3-dihydro-1-benzofuran-2-yl]methanamine,
(+)-{N-methyl-1-[(7-(2,4,6-trichlorophenyl)-2,3-dihydro-1-benzofuran-2-yl]methanamine,
(−)-{N-methyl-1-[(7-(2,4,6-trichlorophenyl)-2,3-dihydro-1-benzofuran-2-yl]methanamine,
(+)-{[7-(2,6-dimethylphenyl)-5-fluoro-2,3-dihydro-1-benzofuran-2-yl]methyl}methylamine,
(−)-{[7-(2,6-dimethylphenyl)-5-fluoro-2,3-dihydro-1-benzofuran-2-yl]methyl}methylamine,
(−)-{[7-(2,6-dimethylphenyl)-5-methoxy-2,3-dihydro-1-benzofuran-2-yl]methyl}methylamine,
(+)-{[7-(2,6-dimethylphenyl)-5-methoxy-2,3-dihydro-1-benzofuran-2-yl]methyl}methylamine,
(+)-{[5-chloro-7-(2,5-dichlorophenyl)-2,3-dihydro-1-benzofuran-2-yl]methyl}methylamine,
(−)-{[5-chloro-7-(2,5-dichlorophenyl)-2,3-dihydro-1-benzofuran-2-yl]methyl}methylamine,
(−)-{[5-chloro-7-(2,6-dimethylphenyl)-2,3-dihydro-1-benzofuran-2-yl]methyl}methylamine,
(+)-{[5-chloro-7-(2,6-dimethylphenyl)-2,3-dihydro-1-benzofuran-2-yl]methyl}methylamine,
(+)-{[7-(2,3-dimethoxyphenyl)-5-methyl-2,3-dihydro-1-benzofuran-2-yl]methyl}methylamine,
(−)-{[7-(2,3-dimethoxyphenyl)-5-methyl-2,3-dihydro-1-benzofuran-2-yl]methyl}methylamine,
(−)-{[7-(2,3-dimethoxyphenyl)-5-fluoro-2,3-dihydro-1-benzofuran-2-yl]methyl}methylamine, or
(+)-{[7-(2,3-dimethoxyphenyl)-5-fluoro-2,3-dihydro-1-benzofuran-2-yl]methyl}methylamine;
or a pharmaceutically acceptable salt thereof.

Compounds of formula I for use in accordance with the present invention may be obtained or produced according to any available means including methods described in detail in U.S. patent application Ser. No. 10/970,714, filed Oct. 21, 2004, U.S. provisional patent application Ser. No. 60/621,023, filed Oct. 21, 2004, and 60/621,024, filed Oct. 21, 2004, and PCT publication number WO2005/044812, the entirety of each of which is hereby incorporated herein by reference.

Without wishing to be bound by any particular theory, the present inventors note that compounds of formula I are highly specific agonists of the 5HT_{2C} receptor. The present invention encompasses the recognition that this unique affinity and selectivity dispayed by compounds of formula I renders them particularly useful for modulation of bladder control, and particularly for treatment of urinary incontinence.

2. Pharmaceutical Compositions

Compounds of formula I may be administered neat in order to modulate bladder activity in accordance with the present invention. More commonly, however, they are administered in the context of a pharmaceutical composition, that contains a therapeutically effective amount of one or more compound of formula I together with one or more other ingredients known to those skilled in the art for formulating pharmaceutical compositions.

As used herein, the terms “pharmaceutically effective amount” or “therapeutically effective amount” mean the total amount of each active component of the pharmaceutical composition or method that is sufficient to show a meaningful patient benefit, i.e., treatment, prevention or amelioration of urinary incontinence or the excessive or undesirable urge to urinate, or a decrease in the frequency of incidence of urinary incontinence. When applied to an individual active ingredient, administered alone, the term refers to that ingredient alone. When applied to a combination, the term refers to combined amounts of the active ingredients that result in the therapeutic effect, whether administered in combination, serially or simultaneously.

In certain embodiments of the invention, compounds of formula I are administered with a daily dose in the range of about 0.5 to about 500 mg, or about 1 mg to about 500 mg. Doses may be administered as a single regimen, such as only prior to bedtime or before travel, or as a continuous regimen divided by two or more doses over the course of a day. The dosage levels and other dosage levels herein are for the average human subject having a weight range of about 65 to 70 kg. The skilled person will readily be able to determine the dosage levels required for a subject whose weight falls outside this range, such as children and the elderly.

The dosage of the combination of the invention in such formulations will depend on its potency, but can be expected to be in the range of from 1 to 500 mg of 5-HT_{2C} receptor agonist for administration up to three times a day. In some embodiments, the dose may be in the range of about 10 to 100 mg (e.g. 10, 25, 50 and 100 mg) of 5-HT_{2C} receptor agonist which can be administered once, twice or three times a day (preferably once). However the precise dose will be as determined by the prescribing physician and will depend on the age and weight of the subject and severity of the symptoms.

Additional ingredients useful in preparing pharmaceutical compositions in accordance with the present invention include, for example, carriers (e.g., in solid or liquid form), flavoring agents, lubricants, solubilizers, suspending agents, fillers, glidants, compression aids, binders, tablet-disintegrating agents, encapsulating materials, emulsifiers, buffers, preservatives, sweeteners, thickening agents, coloring agents, viscosity regulators, stabilizers or osmo-regulators, or combinations thereof.

Solid pharmaceutical compositions preferably contain one or more solid carriers, and optionally one or more other additives such as flavoring agents, lubricants, solubilizers, suspending agents, fillers, glidants, compression aids, binders or tablet-disintegrating agents or an encapsulating material. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes or ion exchange resins, or combinations thereof. In powder pharmaceutical compositions, the carrier is preferably a finely divided solid which is in admixture with the finely divided active ingredient. In tablets, the active ingredient is generally mixed with a carrier having the necessary compression properties in suitable proportions, and optionally, other additives, and compacted into the desired shape and size. Solid pharmaceutical compositions, such as powders and tablets, preferably contain up to 99% of the active ingredient.

Liquid pharmaceutical compositions preferably contain one or more compounds of formula I and one or more liquid carriers to form solutions, suspensions, emulsions, syrups, elixirs, or pressurized compositions. Pharmaceutically acceptable liquid carriers include, for example water, organic solvents, pharmaceutically acceptable oils or fat, or combinations thereof. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators, or combinations thereof. If the liquid formulation is intended for pediatric use, it is generally desirable to avoid inclusion of alcohol.

Examples of liquid carriers suitable for oral or parenteral administration include water (preferably containing additives such as cellulose derivatives such as sodium carboxymethyl cellulose), alcohols or their derivatives (including monohydric alcohols or polyhydric alcohols such as glycols) or oils (e.g., fractionated coconut oil and arachis oil). For parenteral administration the carrier can also be an oily ester such as ethyl oleate and isopropyl myristate. The liquid carrier for pressurized compositions can be halogenated hydrocarbons or other pharmaceutically acceptable propellant.

Liquid pharmaceutical compositions which are sterile solutions or suspensions can be administered parenterally, for example by, intramuscular, intraperitoneal, epidural, intrathecal, intravenous or subcutaneous injection. Pharmaceutical compositions for oral or transmucosal administration may be either in liquid or solid composition form.

In some embodiments of the invention, pharmaceutical compositions are provided in unit dosage form, such as tablets or capsules. In such form, the composition is sub-divided in unit dose containing appropriate quantities of the active ingredient(s). The unit dosage forms can be packaged compositions, for example packeted powders, vials, ampoules, pre-filled syringes or sachets containing liquids. The unit dosage form can be, for example, a capsule or tablet itself, or it can be an appropriate number of any such compositions in package form.

Thus, the present invention also provides a pharmaceutical composition in unit dosage form for modulating bladder activity in a mammal, where the composition contains a therapeutically effective unit dosage of at least one compound of formula I. As one skilled in the art will recognize, the preferred therapeutically effective unit dosage will depend on for example the method of administration. For example, a unit dosage for oral administration often ranges from about 0.5 mg to about 500 mg and more typically from about 1 mg to about 500 mg of the compund of formula I.

The present invention also provides a therapeutic package for dispensing the compounds of formula I to a mammal being treated for pain. In some embodiments, the therapeutic package contains one or more unit dosages of the compound of formula I, a container containing the one or more unit dosages, and labeling directing the use of the package for treating pain in a mammal. In certain embodiments, the unit dose is in tablet or capsule form. In some cases, each unit dosage is a therapeutically effective amount.

3. Other Pharmaceutical Agents

According to the present invention, compounds of formula I may be administered alone to modulate bladder activity, or alternatively may be administered in combination with (whether simultaneously or sequentially) one or more other pharmaceutical agents useful in the modulation of bladder activity. Alternatively or additionally, the compounds of formula I may be administered in combination with one or more other pharmaceutical agents useful in the treatment or prevention of one or more other symptoms, disorders, or diseases suffered by the individual in need of bladder activity modulation.

Other pharmaceutical agents useful in the modulation of bladder activity, and particularly for treatment, prevention, inhibition, and/or amelioration of urinary incontinence, include, for example, desmopressin acetate (available as DDAVP® Nasal Spray and DDAVP® tablets from Aventis Pharmaceuticals), as well as a desmopressin acetate rhinal tube (available from Ferring Pharmaceuticals Inc.). Other products include, for example, tolterodine tartrate (available as DETROL™ tablets from Pharmacia & Upjohn), oxybutinin chloride (available in the form of DITROPAN® tablets and syrup and DITROPAN XL® extended release tablets from ALZA Pharmaceuticals), propanthaline bromide (available in tablet form from Roxane Laboratories, Inc.), hyoscyamine and hyoscyamine sulfate (available, respectively, as CYSTOPAZ® tablets and CYSTOPAZ-M® timed release capsules from PolyMedica Pharmaceuticals (U.S.A.), Inc.), hyoscyamine hydrobromide, flavoxate HCl (available in URISPAS® 100 mg tablets from ALZA Pharmaceuticals), imipramine HCl (available in 10 mg, 25 mg and 50 mg tablets from Geneva Pharmaceuticals, Inc.), phenylpropanolamine, midodrine HCl (available in 2.5 mg and 5 mg PROAMATINE®V tablets from Shire US Inc.), phenoxybenzamine HCl (available as DIBENZYLINE® capsules from WellSpring Pharmaceuticals Corporation), and prazosin HCl (available in MINIPRESS® capsules from Pfizer Inc.). Each of these medicaments may be administered in the pharmaceutically effective amounts and regimens known in the art, including those listed in the Physicians' Desk Reference, 55 Edition, 2001, published by Medical Economics Company, Inc. at Monvale, N.J. 07645-1742, the relevant portions of which are incorporated herein by reference.

Yet other pharmaceutical agents that can act to modulate bladder activity include, for example, other regulators of the 5HT_{2C} receptor. For example, United States Patent Application 2004/0235856 (previously incorporated herein by reference in its entirety) describes a variety of 5HT_{2C} receptor modulators that are useful in accordance with the practice of the present invention. Additional 5HT_{2C} agonists are exemplified in Bishop et al., Expext Opin. Ther. Patent 13:1691-1705, 2003, the entire contents of which are incorporated herein by reference.

Still other pharmaceutical agents that can act to modulate bladder activity include, for example, modulators of one or more KCNQ potassium channels. In some embodiments of the present invention, the compounds of formula I are administered in conjunction with one or more agonists of KCNQ ⅔ or KCNQ⅗. Such KCNQ modulators include, for example, compounds described in U.S. Pat. No. 5,384,330 and those described in U.S. Pat. No. 5,565,483, as well as those described in United States Patent Application Number 2002/0183395; and United States Patent Application Number 2004/0029949. The entire contents of each of these patents and patent applications is incorporated herein by reference. In some embodiments of the present invention, the compounds of formula I are administered with retigabine.

In some embodiments of the present invention, compounds of formula I are administered in conjunction with one or more compounds which act as vasopressin agonists including, but not limited to those described in U.S. Pat. No. 6,194,407 (Failli et al.), U.S. Pat. No. 6,090,803 (Failli et al.), U.S. Pat. No. 6,096,736 (Ogawa et al.), and U.S. Pat. No. 6,096,735 (Ogawa et al.).

In general, it will often be desirable in accordance with the present invention to administer one or more compounds of formula I in conjunction with one or more alpha-adrenergic receptor agonists and/or one or more other sympathomimetic drugs.

Other pharmaceutical agents that may be usefully administered in conjuction with one or more compounds of formula I according to the present invention include agents useful in the treatment of any other symptom, disorder, disease, or medical condition present in the individual being treated, whether related or unrelated to the individual's bladder control issues. Examples of such pharmaceutical agents include, for example, anti-angiogenic agents, anti-neoplastic agents, anti-diabetic agents, anti-infective agents, or gastrointestinal agents, pain management agents, or combinations thereof.

An exemplary listing of pharmaceutically active agents that may be administered in conjunction with one or more compounds of formula I in accordance with the present invention can be found in the Physicians' Desk Reference, 55 Edition, 2001, published by Medical Economics Co., Inc., Montvale, N.J. For many of these listed agents, pharmaceutically effective dosages and regimens are known in the art; many are presented in the Physicians' Desk Reference itself.

4. Uses

The methods of this invention are useful for inducing, assisting or maintaining desirable bladder control in a mammal. The methods are particularly useful for treating a mammal that is experiencing or susceptible to bladder instability or urinary incontinence. Inventive methods include prevention, treatment or inhibition of bladder-related urinary conditions and bladder instability, including idiopathic bladder instability, nocturnal enuresis, nocturia, voiding dysfunction and urinary incontinence (including, for example, stress incontinence, urge incontinence, and/or mixed incontinence). Also treatable or preventable with the methods of this invention is bladder instability secondary to prostate hypertrophy, as is a method for enhancing urethral tone and reducing undesirable urine leakage even in an otherwise healthy person. For example, the inventive methods are applicable to alleviating urine leakage often occurring in women during the first year after childbirth.

In other embodiments, the present compounds are useful for treating urine retention or detrusor sphinctor dyssynergia. Patients suffering from urine retention include those suffering from spinal cord injuries or male patients with benign prostatic hyperplasia

According to the present invention, compounds of formula I are also useful in promoting the temporary delay of urination whenever desirable. The compounds of formula I may be utilized in accordance with the present invention to stabilize the bladder in any applicable context. Inventive methods therefore may be utilized to allow a recipient to control the urgency and frequency of urination.

In some embodiments of the invention, compounds of formula I are administered to a mammal in need thereof for the treatment, prevention, inhibition and/or amelioration of urge urinary incontinence (also known as bladder instability, neurogenic bladder, voiding dysfunction, hyperactive bladder, detrusor overactivity, detrusor hyper-reflexia or uninhibited bladder) or mixed urinary incontinence. Inventive uses include, but are not limited to, those for bladder activities and instabilities in which the urinary urgency is associated with prostatitis, prostatic hypertrophy, interstitial cystitis, urinary tract infections or vaginitis. The methods of this invention may also be used to assist in inhibition or correction of the conditions of Frequency-Urgency Syndrome, and lazy bladder, also known as infrequent voiding syndrome.

The methods of this invention may also be used to treat, prevent, inhibit, or limit the urinary incontinence, urinary instability or urinary urgency associated with or resulting from administrations of other medications, including diuretics, vasopressin antagonists, anticholinergic agents, sedatives or hypnotic agents, narcotics, alpha-adrenergic agonists, alpha-adrenergic antagonists, or calcium channel blockers.

The methods of this invention are useful for inducing or assisting in urinary bladder control or preventing or treating the maladies described herein in humans in need of such relief, including adult and pediatric uses. They may also be utilized for veterinary applications, particularly including canine and feline bladder control methods. If desired, the methods herein may also be used with farm animals, such as ovine, bovine, porcine and equine breeds.

Inventive methods involve delivery of compounds of formula I via any appropriate route of administration including, for example, oral, buccal, sublingual, rectal, nasal, parenteral, intravenous, or other modes. In general, the compounds may be formulated for immediate, delayed, modified, sustained, pulsed, or controlled-release delivery.

For inventive methods utilizing oral delivery, such delivery may be accomplished using solid or liquid formulations, for example in the form of tablets, capsules, multi-particulates, gels, films, ovules, elixirs, solutions or suspensions. In certain embodiments, the compounds are administered as oral tablets or capsules or neat compound or powdered or granular pharmaceutical formulations. Such preparations may be mixed chewable or liquid formulations or food materials or liquids if desirable, for example to facilitate administration to children, to individuals whose ability to swallow tablets is compromised, or to animals. Examples of oral formulations contained in hard gelatin capsules can include those in which the active compound comprises from about 45% to 50%, by weight, of the formulation. Microcrystalline cellulose comprises from about 43% to about 47%, povidone comprises from about 3% to about 4%, and silicon dioxide and magnesium stearate each comprise from about 0.3% to about 0.7%, each by weight.

Modified release and pulsatile release oral dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, camauba wax, paraffin wax, cellulose acetate phthalate, hydroxypropylmethyl cellulose phthalate, methacrylic acid copolymer and mixtures thereof. Modified release and pulsatile release oral dosage forms may contain one or a combination of release rate modifying excipients. Release rate modifying excipients may be present both within the dosage form i.e., within the matrix, and/or on the dosage form, i.e., upon the surface or coating.

Fast dispersing or dissolving dosage oral formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

For inventive methods utilizing intravenous delivery, such administration may be, for example, intracavernous, intravenous, intra-arterial, intraperitoneal, intrathecal, intraventricular, intraurethral, intrasternal, intracranial, intramuscular or subcutaneous, or via by infusion or needleless injection techniques. For such parenteral administration, the compounds of formula I may be prepared and maintained in conventional lyophylized formulations and reconstituted prior to administration with an intravenously acceptable saline solution, such as a 0.9% saline solution. The pH of the intravenous formulation can be adjusted, as is known in the art, with an intravenous and pharmaceutically acceptable acid, such as methanesulfonic acid.

The compounds of formula I can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the compounds of the invention and a suitable powder base such as lactose or starch.

Aerosol or dry powder formulations are preferably arranged so that each metered dose or “puff” contains from 1 .mu.g to 50 mg of a compound of the invention for delivery to the patient. The overall daily dose with an aerosol will be in the range of from 1 .mu.g to 50 mg which may be administered in a single dose or, more usually, in divided doses throughout the day.

Alternatively, the compounds of formula I can be administered in the form of a suppository or pessary, or they may be applied topically in the form of a gel, hydrogel, lotion, solution, cream, ointment or dusting powder. The compounds of the invention may also be dermally or transdermally administered, for example, by the use of a skin patch, depot or subcutaneous injection. They may also be administered by the pulmonary or rectal routes.

For application topically to the skin, the compounds of formula I can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The compounds of formula I may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in published international patent applications WO91/11172, WO94/02518 and WO98/55148.

We claim:

1. A method of treating urinary incontinence in a mammal, comprising administering to said mammal a therapeutically effective amount of a compound of formula I: 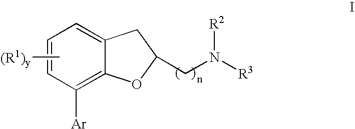
or a pharmaceutically acceptable salt thereof, wherein:
n is one or two;
each of R² and R³ is independently hydrogen, methyl, ethyl, 2-fluoroethyl, 2,2-difluoroethyl or cyclopropyl;
each R¹ is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} substituents;
each R^{x} is independently selected from halogen, OH, lower alkyl, lower alkoxy, lower haloalkyl, lower haloalkoxy, or CN; and
y is 0-3.

2. The method according to claim 1, wherein one of R² and R³ is hydrogen and the other R² and R³ group is hydrogen, methyl, ethyl, 2-fluoroethyl, 2,2-difluoroethyl or cyclopropyl.

3. The method according to claim 2, wherein both of R² and R³ are hydrogen.

4. The method according to claim 1, wherein neither R² and R³ is hydrogen.

5. The method according to claim 1, wherein y is zero.

6. The method according to claim 1, wherein y is other than zero and at least one R¹ group is halogen.

7. The method according to claim 1, wherein y is one and R¹ is halogen, OH, lower alkyl, lower alkoxy, trifluoromethyl, trifluoromethoxy, or CN.

8. The method according to claim 7, wherein y is one and R¹ is fluoro or chloro.

9. The method according to claim 7, wherein said compound is of formula Ia or Ia′: 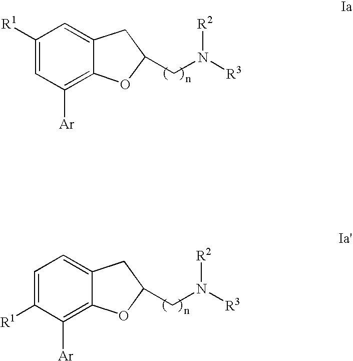
or a pharmaceutically acceptable salt thereof.

10. The method according to claim 1, wherein Ar is unsubstituted phenyl.

11. The method according to claim 1, wherein Ar is phenyl with at least one substituent in the ortho position.

12. The method according to claim 11, wherein Ar is phenyl with at least one substituent in the ortho position selected from halogen, lower alkyl, lower alkoxy, or trifluoromethyl.

13. The method according to claim 11, wherein said compound is of formula Ib or Ic: 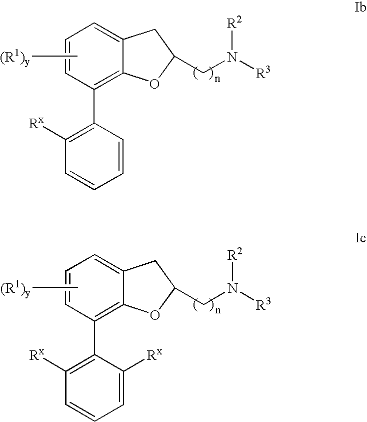
or a pharmaceutically acceptable salt thereof.

14. The method according to claim 13, wherein said compound is of formula Id, Ie, If, Ig, Ih, or Ii: 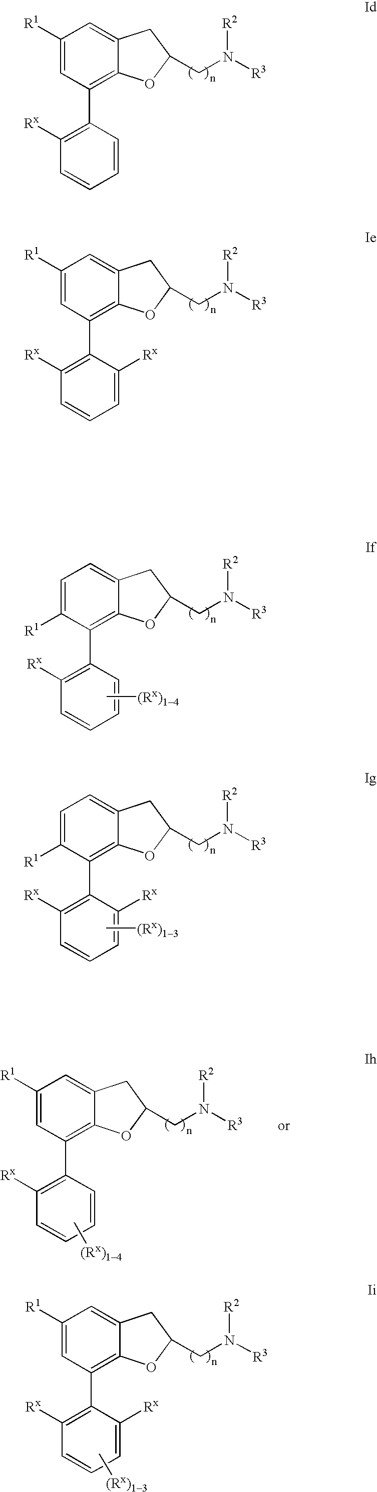
or a pharmaceutically acceptable salt thereof.

15. The method according to claim 1, wherein Ar is selected from:

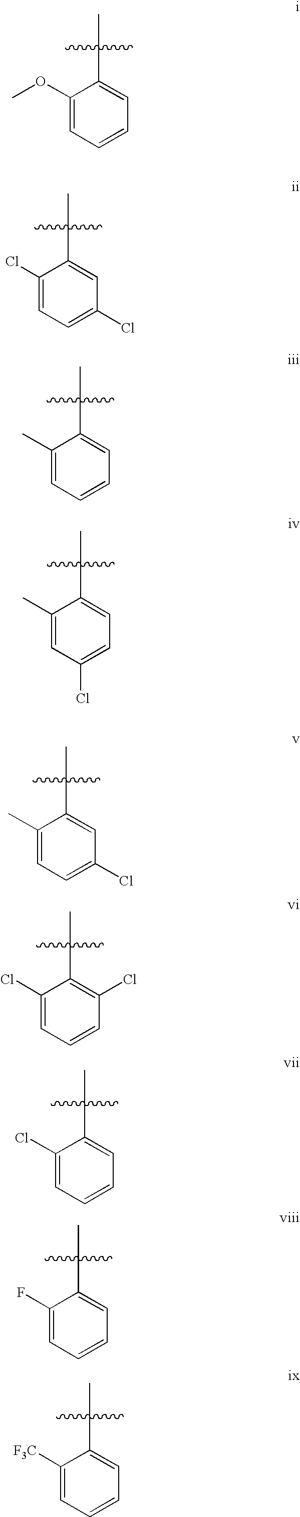

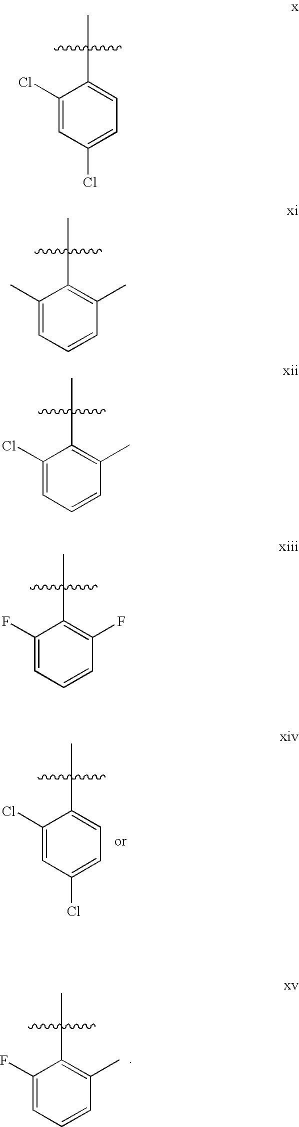

16. The method according to claim 1, wherein said compound is selected from:
(±)-1-{7-[3,5-bis(trifluoromethyl)phenyl]-2,3-dihydro-1-benzofuran-2-yl}methanamine,
(±)-1-[7-(3-chloro-4-fluorophenyl)-2,3-dihydro-1-benzofuran-2-yl]methanamine,
(±)-1-[7-(3,5-dichlorophenyl)-2,3-dihydro-1-benzofuran-2-yl]methanamine,
(±)-1-(7-phenyl-2,3-dihydro-1-benzofuran-2-yl)methanamine,
(+)-(1-(7-phenyl-2,3-dihydro-1-benzofuran-2-yl)methanamine,
(−)-1-(7-phenyl-2,3-dihydro-1-benzofuran-2-yl)methanamine,
(±)-1-[7-(3-methylphenyl)-2,3-dihydro-1-benzofuran-2-yl]methanamine,
(+)-1-[7-(3-methylphenyl)-2,3-dihydro-1-benzofuran-2-yl]methanamine,
(−)-1-[7-(3-methylphenyl)-2,3-dihydro-1-benzofuran-2-yl]methanamine,
(±)-1-(7-thien-3-yl-2,3-dihydro-1-benzofuran-2-yl)methanamine,
(+)-1-(7-thien-3-yl-2,3-dihydro-1-benzofuran-2-yl)methanamine,
(−)-1-(7-thien-3-yl-2,3-dihydro-1-benzofuran-2-yl)methanamine,
(±)-1-[7-(2-methylphenyl)-2,3-dihydro-1-benzofuran-2-yl]methanamine,
(+)-1-[7-(2-methylphenyl)-2,3-dihydro-1-benzofuran-2-yl]methanamine,
(−)-1-[7-(2-methylphenyl)-2,3-dihydro-1-benzofuran-2-yl]methanamine,
(±)-1-[7-(2-fluorophenyl)-2,3-dihydro-1-benzofuran-2-yl]methanamine,
(+)-1-[7-(2-fluorophenyl)-2,3-dihydro-1-benzofuran-2-yl]methanamine,
(−)-1-[7-(2-fluorophenyl)-2,3-dihydro-1-benzofuran-2-yl]methanamine,
(±)-1-{7-[2-(trifluoromethyl)phenyl]-2,3-dihydro-1-benzofuran-2-yl}methanamine,
(−)-1-{7-[2-(trifluoromethyl)phenyl]-2,3-dihydro-1-benzofuran-2-yl}methanamine,
(+)-1-{7-[2-(trifluoromethyl)phenyl]-2,3-dihydro-1-benzofuran-2-yl}methanamine,
(±)-1-[7-(2,6-dimethylphenyl)-2,3-dihydro-1-benzofuran-2-yl]methanamine,
(−)-1-[7-(2,6-dimethylphenyl)-2,3-dihydro-1-benzofuran-2-yl]methanamine,
(+)-1-[7-(2,6-dimethylphenyl)-2,3-dihydro-1-benzofuran-2-yl]methanamine,
(±)-1-[7-(2-methoxyphenyl)-2,3-dihydro-1-benzofuran-2-yl]methanamine,
(±)-1-[7-(2-chlorophenyl)-2,3-dihydro-1-benzofuran-2-yl]methanamine,
(±)-1-[7-(3-fluorophenyl)-2,3-dihydro-1-benzofuran-2-yl]methanamine,
(±)-1-[7-(3-chlorophenyl)-2,3-dihydro-1-benzofuran-2-yl]methanamine,
(+)-1-[7-(3-chlorophenyl)-2,3-dihydro-1-benzofuran-2-yl]methanamine,
(−)-1-[7-(3-chlorophenyl)-2,3-dihydro-1-benzofuran-2-yl]methanamine,
(±)-1-[7-(3-methoxyphenyl)-2,3-dihydro-1-benzofuran-2-yl]methanamine,
(±)-1-{7-[3-(trifluoromethyl)phenyl]-2,3-dihydro-1-benzofuran-2-yl}methanamine,
(±)-1-[7-(4-methylphenyl)-2,3-dihydro-1-benzofuran-2-yl]methanamine,
(+)-1-[7-(4-methylphenyl)-2,3-dihydro-1-benzofuran-2-yl]methanamine,
(−)-1-[7-(4-methylphenyl)-2,3-dihydro-1-benzofuran-2-yl]methanamine,
(±)-1-[7-(4-fluorophenyl)-2,3-dihydro-1-benzofuran-2-yl]methanamine,
(+)-1-[7-(4-fluorophenyl)-2,3-dihydro-1-benzofuran-2-yl]methanamine,
(−)-1-[7-(4-fluorophenyl)-2,3-dihydro-1-benzofuran-2-yl]methanamine,
(±)-1-[7-(4-chlorophenyl)-2,3-dihydro-1-benzofuran-2-yl]methanamine,
(+)-1-[7-(4-chlorophenyl)-2,3-dihydro-1-benzofuran-2-yl]methanamine,
(−)-1-[7-(4-chlorophenyl)-2,3-dihydro-1-benzofuran-2-yl]methanamine,
(±)-1-[7-(4-methoxyphenyl)-2,3-dihydro-1-benzofuran-2-yl]methanamine,
(+)-1-[7-(4-methoxyphenyl)-2,3-dihydro-1-benzofuran-2-yl]methanamine,
(−)-1-[7-(4-methoxyphenyl)-2,3-dihydro-1-benzofuran-2-yl]methanamine,
(±)-1-{7-[4-(trifluoromethyl)phenyl]-2,3-dihydro-1-benzofuran-2-yl}methanamine,
(±)-1-[7-(2,4-dichlorophenyl)-2,3-dihydro-1-benzofuran-2-yl]methanamine,
(±)-1-(5-chloro-7-phenyl-2,3-dihydro-1-benzofuran-2-yl)methanamine,
(+)-1-(5-chloro-7-phenyl-2,3-dihydro-1-benzofuran-2-yl)methanamine,
(−)-1-(5-chloro-7-phenyl-2,3-dihydro-1-benzofuran-2-yl)methanamine,
(±)-1-[5-chloro-7-(2-chlorophenyl)-2,3-dihydro-1-benzofuran-2-yl]methanamine,
(±)-1-[5-chloro-7-(3-methylphenyl)-2,3-dihydro-1-benzofuran-2-yl]methanamine,
(±)-1-[5-chloro-7-(3-chlorophenyl)-2,3-dihydro-1-benzofuran-2-yl]methanamine,
(±)-(5-chloro-7-thien-3-yl-2,3-dihydro-1-benzofuran-2-yl)methylamine,
(−)-(5-chloro-7-thien-3-yl-2,3-dihydro-1-benzofuran-2-yl)methylamine,
(+)-(5-chloro-7-thien-3-yl-2,3-dihydro-1-benzofuran-2-yl)methylamine,
(+)-N-[(5-chloro-7-thien-3-yl-2,3-dihydro-1-benzofuran-2-yl)methyl]-N-methylamine,
(−)-N-[(5-chloro-7-thien-3-yl-2,3-dihydro-1-benzofuran-2-yl)methyl]-N-methylamine,
(±)-1-[5-chloro-7-(2-methylphenyl)-2,3-dihydro-1-benzofuran-2-yl]methanamine,
(+)-1-[5-chloro-7-(2-methylphenyl)-2,3-dihydro-1-benzofuran-2-yl]methanamine,
(−)-1-[5-chloro-7-(2-methylphenyl)-2,3-dihydro-1-benzofuran-2-yl]methanamine,
(±)-1-(4-fluoro-7-phenyl-2,3-dihydro-1-benzofuran-2-yl)methanamine,
(±)-1-[4-fluoro-7-(2-methylphenyl)-2,3-dihydro-1-benzofuran-2-yl]methanamine,
(±)-1-[7-(2-chlorophenyl)-5-fluoro-2,3-dihydro-1-benzofuran-2-yl]methanamine,
(+)-1-[7-(2-chlorophenyl)-5-fluoro-2,3-dihydro-1-benzofuran-2-yl]methanamine,
(−)-1-[7-(2-chlorophenyl)-5-fluoro-2,3-dihydro-1-benzofuran-2-yl]methanamine,
(±)-1-(5-fluoro-7-phenyl-2,3-dihydro-1-benzofuran-2-yl)methanamine,
(±)-1-[5-fluoro-7-(2-methylphenyl)-2,3-dihydro-1-benzofuran-2-yl]methanamine,
(+)-1-[5-fluoro-7-(2-methylphenyl)-2,3-dihydro-1-benzofuran-2-yl]methanamine,
(−)-1-[5-fluoro-7-(2-methylphenyl)-2,3-dihydro-1-benzofuran-2-yl]methanamine,
(±)-1-[5-fluoro-7-(2-fluorophenyl)-2,3-dihydro-1-benzofuran-2-yl]methanamine,
(±)-1-{5-fluoro-7-[2-(trifluoromethyl)phenyl]-2,3-dihydro-1-benzofuran-2-yl}methanamine,
(±)-(4,5-difluoro-7-phenyl-2,3-dihydro-1-benzofuran-2-yl)methylamine,
(±)-1-[4,5-difluoro-7-(2-methylphenyl)-2,3-dihydro-1-benzofuran-2-yl]methanamine,
(±)-1-(5-chloro-2-methyl-7-phenyl-2,3-dihydro-1-benzofuran-2-yl)methanamine,
(±)-(5-chloro-2-methyl-7-thien-3-yl-2,3-dihydro-1-benzofuran-2-yl)methylamine,
(±)-(5-chloro-2-methyl-7-thien-2-yl-2,3-dihydro-1-benzofuran-2-yl)methylamine,
(+)-1-[7-(2-methoxyphenyl)-2,3-dihydro-1-benzofuran-2-yl]methanamine,
(−)-1-[7-(2-chlorophenyl)-2,3-dihydro-1-benzofuran-2-yl]methanamine,
(+)-1-[7-(2-chlorophenyl)-2,3-dihydro-1-benzofuran-2-yl]methanamine,
(+)-1-[7-(3-fluorophenyl)-2,3-dihydro-1-benzofuran-2-yl]methanamine,
(−)-1-[7-(3-fluorophenyl)-2,3-dihydro-1-benzofuran-2-yl]methanamine,
(+)-1-[7-(3-methoxyphenyl)-2,3-dihydro-1-benzofuran-2-yl]methanamine,
(−)-1-[7-(3-methoxyphenyl)-2,3-dihydro-1-benzofuran-2-yl]methanamine,
(−)-1-{7-[3-(trifluoromethyl)phenyl]-2,3-dihydro-1-benzofuran-2-yl}methanamine,
(+)-1-{7-[3-(trifluoromethyl)phenyl]-2,3-dihydro-1-benzofuran-2-yl}methanamine,
(+)-1-{7-[4-(trifluoromethyl)phenyl]-2,3-dihydro-1-benzofuran-2-yl}methanamine,
(−)-1-{7-[4-(trifluoromethyl)phenyl]-2,3-dihydro-1-benzofuran-2-yl}methanamine,
(±)-1-[7-(2,6-difluorophenyl)-2,3-dihydro-1-benzofuran-2-yl]methanamine,
(±)-1-[7-(2,6-dichlorophenyl)-2,3-dihydro-1-benzofuran-2-yl]methanamine,
(−)-1-[7-(2,6-dichlorophenyl)-2,3-dihydro-1-benzofuran-2-yl]methanamine,
(+)-1-[7-(2,6-dichlorophenyl)-2,3-dihydro-1-benzofuran-2-yl]methanamine,
(±)-1-[7-(2,4-dimethoxyphenyl)-2,3-dihydro-1-benzofuran-2-yl]methanamine,
(−)-1-[7-(2,4-dimethoxyphenyl)-2,3-dihydro-1-benzofuran-2-yl]methanamine,
(+)-1-[7-(2,4-dimethoxyphenyl)-2,3-dihydro-1-benzofuran-2-yl]methanamine,
(±)-1-[7-(2,4-difluorophenyl)-2,3-dihydro-1-benzofuran-2-yl]methanamine,
(+)-1-[7-(2,4-difluorophenyl)-2,3-dihydro-1-benzofuran-2-yl]methanamine,
(−)-1-[7-(2,4-difluorophenyl)-2,3-dihydro-1-benzofuran-2-yl]methanamine,
(−)-1-[7-(2,4-dichlorophenyl)-2,3-dihydro-1-benzofuran-2-yl]methanamine,
(+)-1-[7-(2,4-dichlorophenyl)-2,3-dihydro-1-benzofuran-2-yl]methanamine,
(−)-1-{5-fluoro-7-[2-(trifluoromethyl)phenyl]-2,3-dihydro-1-benzofuran-2-yl}methanamine,
(+)-1-{5-fluoro-7-[2-(trifluoromethyl)phenyl]-2,3-dihydro-1-benzofuran-2-yl}methanamine,
(±)-1-[7-(2,3-dimethylphenyl)-2,3-dihydro-1-benzofuran-2-yl]methanamine,
(±)-{[7-(2,3-dimethoxyphenyl)-2,3-dihydro-1-benzofuran-2-yl]methyl}amine,
(−)-{[(7-(2,3-dimethoxyphenyl)-2,3-dihydro-1-benzofuran-2-yl]methyl}amine,
(+)-{[(7-(2,3-dimethoxyphenyl)-2,3-dihydro-1-benzofuran-2-yl]methyl}amine,
(+)-{[7-(4-chloro-2-methylphenyl)-2,3-dihydro-1-benzofuran-2-yl]methyl}amine,
(−)-{[7-(4-chloro-2-methylphenyl)-2,3-dihydro-1-benzofuran-2-yl]methyl) amine,
(±)-{[7-(2,3-difluorophenyl)-2,3-dihydro-1-benzofuran-2-yl]methyl}amine,
(±)-{[7-(2,5-dimethylphenyl)-2,3-dihydro-1-benzofuran-2-yl]methyl}amine,
(±)-{[7-(2,5-dimethoxyphenyl)-2,3-dihydro-1-benzofuran-2-yl]methyl}amine,
(±)-{[7-(2,5-dichlorophenyl)-2,3-dihydro-1-benzofuran-2-yl]methyl}amine,
(+)-{[7-(2,5-dichlorophenyl)-2,3-dihydro-1-benzofuran-2-yl]methyl}amine,
(−)-{[7-(2,5-dichlorophenyl)-2,3-dihydro-1-benzofuran--yl]methyl}amine,
(±)-{[7-(2,4,6-trichlorophenyl)-2,3-dihydro-1-benzofuran-2-yl]methyl}amine,
(±)-{[7-(4-chloro-2-methylphenyl)-2,3-dihydro-1-benzofuran-2-yl]methyl}amine,
(±)-{[7-(5-chloro-2-methylphenyl)-2,3-dihydro-1-benzofuran-2-yl]methyl}amine,
(═)-{[7-(5-chloro-2-methoxyphenyl)-2,3-dihydro-1-benzofuran-2-yl]methyl}amine,
(±)-[(7-pyridin-3-yl-2,3-dihydro-1-benzofuran-2-yl)methyl]amine,
(+)-{[7-pyridin-3-yl-2,3-dihydro-1-benzofuran-2-yl]methyl}amine,
(−)-{[7-pyridin-3-yl-2,3-dihydro-1-benzofuran-2-yl]methyl}amine,
(±)-{[5-fluoro-7-(2-methoxyphenyl)-2,3-dihydro-1-benzofuran-2-yl]methyl}amine,
(±)-{[5-fluoro-7-(3-fluorophenyl)-2,3-dihydro-1-benzofuran-2-yl]methyl}amine,
(±)-{[5-fluoro-7-(3-methoxyphenyl)-2,3-dihydro-1-benzofuran-2-yl]methyl}amine,
(±)-{[5-fluoro-7-(3-methylphenyl)-2,3-dihydro-1-benzofuran-2-yl]methyl}amine,
(±)-{[5-fluoro-7-(4-fluorophenyl)-2,3-dihydro-1-benzofuran-2-yl]methyl}amine,
(±)-{[5-fluoro-7-(4-chlorophenyl)-2,3-dihydro-1-benzofuran-2-yl]methyl}amine,
(±)-{[5-fluoro-7-(4-methylphenyl)-2,3-dihydro-1-benzofuran-2-yl]methyl}amine,
(±)-{[5-fluoro-7-(4-methoxyphenyl)-2,3-dihydro-1-benzofuran-2-yl]methyl}amine,
(±)-[(5-fluoro-7-thien-3-yl-2,3-dihydro-1-benzofuran-2-yl)methyl]amine,
(±)-{[5-fluoro-7-(3-furyl)-2,3-dihydro-1-benzofuran-2-yl]methyl}amine,
(±)-[(5-fluoro-7-pyridin-2-yl-2,3-dihydro-1-benzofuran-2-yl)methyl]amine,
(±)-[(5-fluoro-7-pyridin-3-yl-2,3-dihydro-1-benzofuran-2-yl)methyl]amine,
(−)-[(5-fluoro-7-pyridin-3-yl-2,3-dihydro-1-benzofuran-2-yl)methyl]amine,
(+)-{[5-fluoro-7-pyridin-3-yl-2,3-dihydro-1-benzofuran-2-yl]methyl}amine,
(±)-[(5-fluoro-7-pyridin-4-yl-2,3-dihydro-1-benzofuran-2-yl)methyl]amine,
(±)-[(5-fluoro-7-pyrimidin-5-yl-2,3-dihydro-1-benzofuran-2-yl)methyl]amine,
(±)-{[7-(2,3-dichlorophenyl)-5-fluoro-2,3-dihydro-1-benzofuran-2-yl]methyl}amine,
(±)-{[7-(2,3-dimethoxyphenyl)-5-fluoro-2,3-dihydro-1-benzofuran-2-yl]methyl}amine,
(−)-{[7-(2,3-dimethoxyphenyl)-5-fluoro-2,3-dihydro-1-benzofuran-2-yl]methyl}amine,
(+)-{[7-(2,3-dimethoxyphenyl)-5-fluoro-2,3-dihydro-1-benzofuran-2-yl]methyl}amine,
(±)-{[7-(2,4-difluorophenyl)-5-fluoro-2,3-dihydro-1-benzofuran-2-yl]methyl}amine,
(±)-{[7-(2,4-dichlorophenyl)-5-fluoro-2,3-dihydro-1-benzofuran-2-yl]methyl}amine,
(−)-{[5-fluoro-7-(2,4-dichlorophenyl)-2,3-dihydro-1-benzofuran-2-yl]methyl}amine,
(+)-{[5-fluoro-7-(2,4-dichlorophenyl)-2,3-dihydro-1-benzofuran-2-yl]methyl}amine,
(±)-{[7-(2,4-dimethoxyphenyl)-5-fluoro-2,3-dihydro-1-benzofuran-2-yl]methyl}amine,
(±)-{[7-(2,5-difluorophenyl)-5-fluoro-2,3-dihydro-1-benzofuran-2-yl]methyl}amine,
(±)-{[7-(2,5-dichlorophenyl)-5-fluoro-2,3-dihydro-1-benzofuran-2-yl]methyl}amine,
(±)-{[7-(2,5-dimethylphenyl)-5-fluoro-2,3-dihydro-1-benzofuran-2-yl]methyl}amine,
(±)-{[7-(2,5-dimethoxyphenyl)-5-fluoro-2,3-dihydro-1-benzofuran-2-yl]methyl}amine,
(±)-{[5-fluoro-7-(5-methoxy-2-methylphenyl)-2,3-dihydro-1-benzofuran-2-yl]methyl}amine,
(±)-{[5-fluoro-7-(2-methoxy-5-methylphenyl)-2,3-dihydro-1-benzofuran-2-yl]methyl}amine,
(±)-{[7-(2,6-difluorophenyl)-5-fluoro-2,3-dihydro-1-benzofuran-2-yl]methyl}amine,
(±)-{[7-(2,6-dimethylphenyl)-5-fluoro-2,3-dihydro-1-benzofuran-2-yl]methyl}amine,
(+)-{[7-(2,6-dimethylphenyl)-5-fluoro-2,3-dihydro-1-benzofuran-2-yl]methyl}amine,
(−)-{[7-(2,6-dimethylphenyl)-5-fluoro-2,3-dihydro-1-benzofuran-2-yl]methyl}amine,
(±)-{[7-(2,6-dichlorophenyl)-5-fluoro-2,3-dihydro-1-benzofuran-2-yl]methyl}amine,
(±)-N-{[7-(2,6-dichlorophenyl)-5-fluoro-2,3-dihydro-1-benzofuran-2-yl]methyl}cyclopropanamine,
(±)-1-cyclopropyl-N-{[7-(2,6-dichlorophenyl)-5-fluoro-2,3-dihydro-1-benzofuran-2-yl]methyl}methanamine,
(±)-N-{[7-(2,6-dichlorophenyl)-5-fluoro-2,3-dihydro-1-benzofuran-2-yl]methyl}ethanamine,
(±)-{[7-(2,6-dichlorophenyl)-5-fluoro-2,3-dihydro-1-benzofuran-2-yl]methyl}dimethylamine,
(±)-{[5-chloro-7-(2-fluorophenyl)-2,3-dihydro-1-benzofuran-2-yl]methyl}amine,
(±)-{[5-chloro-7-(2-methoxyphenyl)-2,3-dihydro-1-benzofuran-2-yl]methyl}amine,
(±)-{[5-chloro-7-(3-furyl)-2,3-dihydro-1-benzofuran-2-yl]methyl}amine,
(±)-{[5-chloro-7-(2,3-difluorophenyl)-2,3-dihydro-1-benzofuran-2-yl]methyl}amine,
(−)-{[5-chloro-7-(2,3-difluorophenyl)-2,3-dihydro-1-benzofuran-2-yl]methyl}amine,
(+)-{[5-chloro-7-(2,3-difluorophenyl)-2,3-dihydro-1-benzofuran-2-yl]methyl}amine,
(±)-{[5-chloro-7-(2,3-dichlorophenyl)-2,3-dihydro-1-benzofuran-2-yl]methyl}amine,
(±)-{[5-chloro-7-(2,3-dimethylphenyl)-2,3-dihydro-1-benzofuran-2-yl]methyl}amine,
(±)-{[5-chloro-7-(2,3-dimethoxyphenyl)-2,3-dihydro-1-benzofuran-2-yl]methyl}amine,
(±)-{[5-chloro-7-(2,4-difluorophenyl)-2,3-dihydro-1-benzofuran-2-yl]methyl}amine,
(±)-{[5-chloro-7-(2,4-dichlorophenyl)-2,3-dihydro-1-benzofuran-2-yl]methyl}amine,
(−)-{[5-chloro-7-(2,4-dichlorophenyl)-2,3-dihydro-1-benzofuran-2-yl]methyl}amine,
(+)-{[5-chloro-7-(2,4-dichlorophenyl)-2,3-dihydro-1-benzofuran-2-yl]methyl}amine,
(±)-{[5-chloro-7-(2,4-dimethoxyphenyl)-2,3-dihydro-1-benzofuran-2-yl]methyl}amine,
(±)-{[5-chloro-7-(2,5-difluorophenyl)-2,3-dihydro-1-benzofuran-2-yl]methyl}amine,
(±)-{[5-chloro-7-(2,5-dichlorophenyl)-2,3-dihydro-1-benzofuran-2-yl]methyl}amine,
(±)-{[5-chloro-7-(5-chloro-2-methoxyphenyl)-2,3-dihydro-1-benzofuran-2-yl]methyl}amine,
(±)-{[5-chloro-7-(3,4-difluorophenyl)-2,3-dihydro-1-benzofuran-2-yl]methyl}amine,
(±)-{[5-chloro-7-(3-chloro-4-fluorophenyl)-2,3-dihydro-1-benzofuran-2-yl]methyl}amine,
(±)-{[5-chloro-7-(2,6-dimethylphenyl)-2,3-dihydro-1-benzofuran-2-yl]methyl}amine,
(−)-{[5-chloro-7-(2,6-dimethylphenyl)-2,3-dihydro-1-benzofuran-2-yl]methyl}amine,
(+)-{[5-chloro-7-(2,6-dimethylphenyl)-2,3-dihydro-1-benzofuran-2-yl]methyl}amine,
(±)-{[5-chloro-7-(2,6-dichlorophenyl)-2,3-dihydro-1-benzofuran-2-yl]methyl}amine,
(+)-{[5-chloro-7-(2,6-dichlorophenyl)-2,3-dihydro-1-benzofuran-2-yl]methyl}amine,
(−)-{[5-chloro-7-(2,6-dichlorophenyl)-2,3-dihydro-1-benzofuran-2-yl]methyl}amine,
(±)-{[(5-chloro-7-pyridin-3-yl-2,3-dihydro-1-benzofuran-2-yl)methyl]amine,
(±)-N-{[5-chloro-7-(2,6-dimethylphenyl)-2,3-dihydro-1-benzofuran-2-yl]methyl}cyclopropanamine,
(±)-N-{[5-chloro-7-(2,6-dimethylphenyl)-2,3-dihydro-1-benzofuran-2-yl]methyl}(cyclopropylmethyl)amine,
(±)-N-{[5-chloro-7-(2,6-dimethylphenyl)-2,3-dihydro-1-benzofuran-2-yl]methyl}ethanamine,
(±)-{[(5-methyl-7-phenyl-2,3-dihydro-1-benzofuran-2-yl)methyl]amine,
(±)-{[7-(2-methylphenyl)-5-methyl-2,3-dihydro-1-benzofuran-2-yl]methyl}amine,
(±)-{[7-(2-fluorophenyl)-5-methyl-2,3-dihydro-1-benzofuran-2-yl]methyl}amine,
(±)-{[7-(2-methoxyphenyl)-5-methyl-2,3-dihydro-1-benzofuran-2-yl]methyl}amine,
(±)-{[7-(2-chlorophenyl)-5-methyl-2,3-dihydro-1-benzofuran-2-yl]methyl}amine,
(±)-({5-methyl-7-[2-(trifluoromethyl)phenyl]-2,3-dihydro-1-benzofuran-2-yl}methyl)amine,
(±)-{[7-(3-chlorophenyl)-5-methyl-2,3-dihydro-1-benzofuran-2-yl]methyl}amine,
(±)-{[7-(3-methylphenyl)-5-methyl-2,3-dihydro-1-benzofuran-2-yl]methyl}amine,
(±)-{[7-(4-methylphenyl)-5-methyl-2,3-dihydro-1-benzofuran-2-yl]methyl}amine,
(±)-{[7-(4-fluorophenyl)-5-methyl-2,3-dihydro-1-benzofuran-2-yl]methyl}amine,
(±)-{[7-(4-chlorophenyl)-5-methyl-2,3-dihydro-1-benzofuran-2-yl]methyl}amine,
(±)-{[7-(4-methoxyphenyl)-5-methyl-2,3-dihydro-1-benzofuran-2-yl]methyl}amine,
(±)-{[7-(2,3-dimethoxyphenyl)-5-methyl-2,3-dihydro-1-benzofuran-2-yl]methyl}amine,
(−)-{[7-(2,3-dimethoxyphenyl)-5-methyl-2,3-dihydro-1-benzofuran-2-yl]methyl}amine,
(+)-{[7-(2,3-dimethoxyphenyl)-5-methyl-2,3-dihydro-1-benzofuran-2-yl]methyl}amine,
(±)-{[7-(2,4-dichlorophenyl)-5-methyl-2,3-dihydro-1-benzofuran-2-yl]methyl}amine,
(−)-{[7-(2,4-dichlorophenyl)-5-methyl-2,3-dihydro-1-benzofuran-2-yl]methyl}amine,
(+)-{[7-(2,4-dichlorophenyl)-5-methyl-2,3-dihydro-1-benzofuran-2-yl]methyl}amine,
(±)-{[7-(2,5-dichlorophenyl)-5-methyl-2,3-dihydro-1-benzofuran-2-yl]methyl}amine,
(±)-{[7-(2,6-dimethylphenyl)-5-methyl-2,3-dihydro-1-benzofuran-2-yl]methyl}amine,
(±)-{[7-(2,6-dichlorophenyl)-5-methyl-2,3-dihydro-1-benzofuran-2-yl]methyl}amine,
(−)-{[7-(2,6-dichlorophenyl)-5-methyl-2,3-dihydro-1-benzofuran-2-yl]methyl}amine,
(+)-{[7-(2,6-dichlorophenyl)-5-methyl-2,3-dihydro-1-benzofuran-2-yl]methyl}amine,
(±)-{[5-ethyl-7-(2-methylphenyl)-2,3-dihydro-1-benzofuran-2-yl]methyl}amine,
(±)-{[5-ethyl-7-(2-chlorophenyl)-2,3-dihydro-1-benzofuran-2-yl]methyl}amine,
(±)-{[5-(trifluoromethyl)-7-(2-fluorophenyl)-2,3-dihydro-1-benzofuran-2-yl]methyl}amine,
(±)-{[5-(trifluoromethyl)-7-(2-methylphenyl)-2,3-dihydro-1-benzofuran-2-yl]methyl}amine,
(±)-{[5-(trifluoromethyl)-7-(2-chlorophenyl)-2,3-dihydro-1-benzofuran-2-yl]methyl}amine,
(±)-{[5-(trifluoromethyl)-7-(2-methoxyphenyl)-2,3-dihydro-1-benzofuran-2-yl]methyl}amine,
(±)-{[5-(trifluoromethyl)-7-(2-(trifluoromethyl)phenyl)-2,3-dihydro-1-benzofuran-2-yl]methyl}amine,
(±)-{[5-(trifluoromethyl)-7-(3-methylphenyl)-2,3-dihydro-1-benzofuran-2-yl]methyl}amine,
(±)-{[5-(trifluoromethyl)-7-(3-fluorophenyl)-2,3-dihydro-1-benzofuran-2-yl]methyl}amine,
(±)-{[5-(trifluoromethyl)-7-(3-chlorophenyl)-2,3-dihydro-1-benzofuran-2-yl]methyl}amine,
(±)-{[5-(trifluoromethyl)-7-(3-methoxyphenyl)-2,3-dihydro-1-benzofuran-2-yl]methyl}amine,
(±)-{[5-(trifluoromethyl)-7-(3-(trifluoromethyl)phenyl)-2,3-dihydro-1-benzofuran-2-yl]methyl}amine,
(±)-{[5-(trifluoromethyl)-7-(4-methylphenyl)-2,3-dihydro-1-benzofuran-2-yl]methyl}amine,
(±)-{[5-(trifluoromethyl)-7-(4-fluorophenyl)-2,3-dihydro-1-benzofuran-2-yl]methyl}amine,
(±)-{[5-(trifluoromethyl)-7-(4-chlorophenyl)-2,3-dihydro-1-benzofuran-2-yl]methyl}amine,
(±)-{[5-(trifluoromethyl)-7-(4-methoxyphenyl)-2,3-dihydro-1-benzofuran-2-yl]methyl}amine,
(±)-{[5-(trifluoromethyl)-7-(4-(trifluoromethyl)phenyl)-2,3-dihydro-1-benzofuran-2-yl]methyl}amine,
(±)-{[7-(2,3-dimethylphenyl)-5-(trifluoromethyl)-2,3-dihydro-1-benzofuran-2-yl]methyl}amine,
(±)-{[7-(2,3-difluorophenyl)-5-(trifluoromethyl)-2,3-dihydro-1-benzofuran-2-yl]methyl}amine,
(±)-{[7-(2,3-dichlorophenyl)-5-(trifluoromethyl)-2,3-dihydro-1-benzofuran-2-yl]methyl}amine,
(±)-{[7-(2,3-dimethoxyphenyl)-5-(trifluoromethyl)-2,3-dihydro-1-benzofuran-2-yl]methyl}amine,
(±)-{[7-(2,4-difluorophenyl)-5-(trifluoromethyl)-2,3-dihydro-1-benzofuran-2-yl]methyl}amine,
(35 )-{[7-(2,4-dimethoxyphenyl)-5-(trifluoromethyl)-2,3-dihydro-1-benzofuran-2-yl]methyl}amine,
(±)-{[7-(3,4-difluorophenyl)-5-(trifluoromethyl)-2,3-dihydro-1-benzofuran-2-yl]methyl}amine,
(±)-{[7-(3-chloro-4-fluorophenyl)-5-(trifluoromethyl)-2,3-dihydro-1-benzofuran-2-yl]methyl}amine,
(±)-{[7-(2,5-difluorophenyl)-5-(trifluoromethyl)-2,3-dihydro-1-benzofuran-2-yl]methyl}amine,
(±)-{[7-(2,5-dichlorophenyl)-5-(trifluoromethyl)-2,3-dihydro-1-benzofuran-2-yl]methyl}amine,
(±)-{[7-(2,6-dimethylphenyl)-5-(trifluoromethyl)-2,3-dihydro-1-benzofuran-2-yl]methyl}amine,
(±)-4-[2-(aminomethyl)-5-(trifluoromethyl)-2,3-dihydro-1-benzofuran-7-yl]benzonitrile
(±)-{[7-(3-furyl)-5-(trifluoromethyl)-2,3-dihydro-1-benzofuran-2-yl]methyl}amine,
(±)-{[7-thien-3-yl-5-(trifluoromethyl)-2,3-dihydro-1-benzofuran-2-yl]methyl}amine,
(±)-{[7-pyridin-3-yl-5-(trifluoromethyl)-2,3-dihydro-1-benzofuran-2-yl]methyl}amine,
(±)-{[7-(2-fluorophenyl)-5-methoxy-2,3-dihydro-1-benzofuran-2-yl]methyl}amine,
(±)-{[7-(2-chlorophenyl)-5-methoxy-2,3-dihydro-1-benzofuran-2-yl]methyl}amine,
(±)-{[7-(2-methylphenyl)-5-methoxy-2,3-dihydro-1-benzofuran-2-yl]methyl}amine,
(±)-{[7-(2-methoxyphenyl)-5-methoxy-2,3-dihydro-1-benzofuran-2-yl]methyl}amine,
(±)-{[5-methoxy-7-(3-thienyl)-2,3-dihydro-1-benzofuran-2-yl]methyl}amine,
(±)-{[7-(2,3-difluorophenyl)-5-methoxy-2,3-dihydro-1-benzofuran-2-yl]methyl}amine,
(±)-{[7-(2,3-dichlorophenyl)-5-methoxy-2,3-dihydro-1-benzofuran-2-yl]methyl}amine,
(±)-{[7-(2,3-dimethylphenyl)-5-methoxy-2,3-dihydro-1-benzofuran-2-yl]methyl}amine,
(±)-{[7-(2,4-difluorophenyl)-5-methoxy-2,3-dihydro-1-benzofuran-2-yl]methyl}amine,
(±)-{[7-(2,4-dichlorophenyl)-5-methoxy-2,3-dihydro-1-benzofuran-2-yl]methyl}amine,
(±)-{[7-(2,5-difluorophenyl)-5-methoxy-2,3-dihydro-1-benzofuran-2-yl]methyl}amine,
(±)-{[7-(2,5-dichlorophenyl)-5-methoxy-2,3-dihydro-1-benzofuran-2-yl]methyl}amine,
(+)-{[7-(2,5-dichlorophenyl)-5-methoxy-2,3-dihydro-1-benzofuran-2-yl]methyl}amine,
(−)-{[7-(2,5-dichlorophenyl)-5-methoxy-2,3-dihydro-1-benzofuran-2-yl]methyl}amine,
(±)-{[7-(2,5-dimethylphenyl)-5-methoxy-2,3-dihydro-1-benzofuran-2-yl]methyl}amine,
(±)-{[7-(2,5-dimethoxylphenyl)-5-methoxy-2,3-dihydro-1-benzofuran-2-yl]methyl}amine,
(±)-{[7-(5-chloro-2-methoxyphenyl)-5-methoxy-2,3-dihydro-1-benzofuran-2-yl]methyl}amine,
(±)-{[7-(3-chloro-4-fluorophenyl)-5-methoxy-2,3-dihydro-1-benzofuran-2-yl]methyl}amine,
(±)-{[7-(2,6-dimethylphenyl)-5-methoxy-2,3-dihydro-1-benzofuran-2-yl]methyl}amine,
(±)-[(N-methyl-1-[7-(2-methoxyphenyl)-2,3-dihydro-1-benzofuran-2-yl]methanamine,
(±)-[(N-methyl-1-[7-(3-methylphenyl)-2,3-dihydro-1-benzofuran-2-yl]methanamine,
(±)-[(N-methyl-1-[7-(3-fluorophenyl)-2,3-dihydro-1-benzofuran-2-yl]methanamine,
(±)-[(N-methyl-1-[7-(3-chlorophenyl)-2,3-dihydro-1-benzofuran-2-yl]methanamine,
(±)-[(N-methyl-1-[7-(3-methoxyphenyl)-2,3-dihydro-1-benzofuran-2-yl]methanamine,
(±)-[(N-methyl-1-[7-(4-methylphenyl)-2,3-dihydro-1-benzofuran-2-yl]methanamine,
(±)-[(N-methyl-1-[7-(4-fluorophenyl)-2,3-dihydro-1-benzofuran-2-yl]methanamine,
(±)-[(N-methyl-1-[7-(4-chlorophenyl)-2,3-dihydro-1-benzofuran-2-yl]methanamine,
(±)-[(N-methyl-1-[7-(4-methoxyphenyl)-2,3-dihydro-1-benzofuran-2-yl]methanamine,
(±)-[(N-methyl-1-[7-(2,3-dimethylphenyl)-2,3-dihydro-1-benzofuran-2-yl]methanamine,
(±)-[(N-methyl-1-[7-(2,3-dimethoxyphenyl)-2,3-dihydro-1-benzofuran-2-yl]methanamine,
(±)-[(N-methyl-1-[7-(2,4-difluorophenyl)-2,3-dihydro-1-benzofuran-2-yl]methanamine,
(±)-[(N-methyl-1-[7-(2,4-dichlorophenyl)-2,3-dihydro-1-benzofuran-2-yl]methanamine,
(±)-[(N-methyl-1-[7-(2,4-dimethoxyphenyl)-2,3-dihydro-1-benzofuran-2-yl]methanamine,
(±)-[(N-methyl-1-[7-(2,5-dimethylphenyl)-2,3-dihydro-1-benzofuran-2-yl]methanamine,
(±)-[(N-methyl-1-[7-(2,5-difluorophenyl)-2,3-dihydro-1-benzofuran-2-yl]methanamine,
(±)-[(N-methyl-1-[7-(2,5-dichlorophenyl)-2,3-dihydro-1-benzofuran-2-yl]methanamine,
(±)-[(N-methyl-1-[7-(5-chloro-2-methoxyphenyl)-2,3-dihydro-1-benzofuran-2-yl]methanamine,
(±)-[(N-methyl-1-[7-(5-chloro-2-methylphenyl)-2,3-dihydro-1-benzofuran-2-yl]methanamine,
(±)-[(N-methyl-1-[7-(2,6-dimethylphenyl)-2,3-dihydro-1-benzofuran-2-yl]methanamine,
(±)-[(N-methyl-1-[7-(2,6-dichlorophenyl)-2,3-dihydro-1-benzofuran-2-yl]methanamine,
(−)-{[7-(2,6-dichlorophenyl)-2,3-dihydro-1-benzofuran-2-yl]methyl}methylamine,
(+)-{[7-(2,6-dichlorophenyl)-2,3-dihydro-1-benzofuran-2-yl]methyl}methylamine,
(±)-N-methyl-1-(7-pyridin-3-yl-2,3-dihydro-1-benzofuran-2-yl)methanamine,
(±)-[(N-methyl-1-[7-(2,3-difluorophenyl)-2,3-dihydro-1-benzofuran-2-yl]methanamine,
(±)-{[5-fluoro-7-(2-fluorophenyl)-2,3-dihydro-1-benzofuran-2-yl]methyl}methylamine,
(±)-{[5-fluoro-7-(2-chlorophenyl)-2,3-dihydro-1-benzofuran-2-yl]methyl}methylamine,
(±)-{[5-fluoro-7-(2-methylphenyl)-2,3-dihydro-1-benzofuran-2-yl]methyl}methylamine,
(−)-{[5-fluoro-7-(2-methylphenyl)-2,3-dihydro-1-benzofuran-2-yl]methyl}methylamine,
(+)-{[5-fluoro-7-(2-methylphenyl)-2,3-dihydro-1-benzofuran-2-yl]methyl}methylamine,
(±)-{[5-fluoro-7-(2-methoxyphenyl)-2,3-dihydro-1-benzofuran-2-yl]methyl}methylamine,
(±)-{[7-(2,3-dimethoxyphenyl)-5-fluoro-2,3-dihydro-1-benzofuran-2-yl]methyl}methylamine,
(±)-[7-(2,4-dichlorophenyl)-5-fluoro-2,3-dihydro-1-benzofuran-2-yl]methyl}methylamine,
(−)-[7-(2,4-dichlorophenyl)-5-fluoro-2,3-dihydro-1-benzofuran-2-yl]methyl}methylamine,
(+)-[7-(2,4-dichlorophenyl)-5-fluoro-2,3-dihydro-1-benzofuran-2-yl]methyl}methylamine,
(±)-{[7-(2,5-difluorophenyl)-5-fluoro-2,3-dihydro-1-benzofuran-2-yl]methyl}methylamine,
(±)-{[7-(2,5-dichlorophenyl)-5-fluoro-2,3-dihydro-1-benzofuran-2-yl]methyl}methylamine,
(+)-{[5-fluoro-7-(2,5-dichlorophenyl)-2,3-dihydro-1-benzofuran-2-yl]methyl}methylamine,
(−)-{[5-fluoro-7-(2,5-dichlorophenyl)-2,3-dihydro-1-benzofuran-2-yl]methyl}methylamine,
(±)-{[7-(2,5-dimethylphenyl)-5-fluoro-2,3-dihydro-1-benzofuran-2-yl]methyl}methylamine,
(±)-{[7-(2,6-dimethylphenyl)-5-fluoro-2,3-dihydro-1-benzofuran-2-yl]methyl}methylamine,
(±)-{[7-(2,6-dichlorophenyl)-5-fluoro-2,3-dihydro-1-benzofuran-2-yl]methyl}methylamine,
(±)-{[5-fluoro-7-(5-methoxy-2-methylphenyl)-2,3-dihydro-1-benzofuran-2-yl]methyl}methylamine,
(±)-{[5-fluoro-7-(2-methoxy-5-methylphenyl)-2,3-dihydro-1-benzofuran-2-yl]methyl}methylamine,
(±)-{[7-(5-chloro-2-methoxyphenyl)-5-fluoro-2,3-dihydro-1-benzofuran-2-yl]methyl}methylamine,
(±)-[(5-fluoro-7-pyridin-3-yl-2,3-dihydro-1-benzofuran-2-yl)methyl]methylamine,
(±)-[(5-chloro-7-(2-methoxyphenyl)-2,3-dihydro-1-benzofuran-2-yl]methyl}methylamine,
(±)-{[5-chloro-7-(2,6-dimethylphenyl)-2,3-dihydro-1-benzofuran-2-yl]methyl}methylamine,
(±)-[(5-chloro-7-(2-fluorophenyl)-2,3-dihydro-1-benzofuran-2-yl]methyl}methylamine,
(±)-{[5-chloro-7-(2,3-difluorophenyl)-2,3-dihydro-1-benzofuran-2-yl]methyl}methylamine,
(±)-{[5-chloro-7-(2,3-dichlorophenyl)-2,3-dihydro-1-benzofuran-2-yl]methyl) methylamine,
(±)-{[5-chloro-7-(2,3-dimethylphenyl)-2,3-dihydro-1-benzofuran-2-yl]methyl}methylamine,
(±)-{[5-chloro-7-(2,3-dimethoxyphenyl)-2,3-dihydro-1-benzofuran-2-yl]methyl}methylamine,
(±)-{[5-chloro-7-(2,4-difluorophenyl)-2,3-dihydro-1-benzofuran-2-yl]methyl) methylamine,
(±)-{[5-chloro-7-(2,4-dichlorophenyl)-2,3-dihydro-1-benzofuran-2-yl]methyl ) methylamine,
(±)-{[5-chloro-7-(2,4-dimethoxyphenyl)-2,3-dihydro-1-benzofuran-2-yl]methyl}methylamine,
(±)-{[5-chloro-7-(2,5-difluorophenyl)-2,3-dihydro-1-benzofuran-2-yl]methyl}methylamine,
(±)-{[5-chloro-7-(2,5-dichlorophenyl)-2,3-dihydro-1-benzofuran-2-yl]methyl}methylamine,
(±)-{[5-chloro-7-(5-chloro-2-methoxyphenyl)-2,3-dihydro-1-benzofuran-2-yl]methyl}methylamine,
(±)-{[5-chloro-7-(3,4-difluorophenyl)-2,3-dihydro-1-benzofuran-2-yl]methyl}methylamine,
(±)-[5-chloro-7-(3-chloro-4-fluorophenyl)-2,3-dihydro-1-benzofuran-2-yl]methyl}methylamine,
(±)-{[7-(2-fluorophenyl)-5-methyl-2,3-dihydro-1-benzofuran-2-yl]methyl}methylamine,
(±)-{[7-(2-chlorophenyl)-5-methyl-2,3-dihydro-1-benzofuran-2-yl]methyl}methylamine,
(±)-{[7-(2-methoxyphenyl)-5-methyl -2,3-dihydro-1-benzofuran-2-yl]methyl}methylamine,
(±)-{[7-(3-methylphenyl)-5-methyl-2,3-dihydro-1-benzofuran-2-yl]methyl) methylamine,
(±)-{[7-(3-chlorophenyl)-5-methyl-2,3-dihydro-1-benzofuran-2-yl]methyl) methylamine,
(±)-{[7-(4-methylphenyl)-5-methyl-2,3-dihydro-1-benzofuran-2-yl]methyl}methylamine,
(±)-{[7-(4-chlorophenyl)-5-methyl-2,3-dihydro-1-benzofuran-2-yl]methyl) methylamine,
(±)-{[7-(4-fluorophenyl)-5-methyl-2,3-dihydro-1-benzofuran-2-yl]methyl}methylamine,
(±)-{[7-(4-methoxyphenyl)-5-methyl-2,3-dihydro-1-benzofuran-2-yl]methyl) methylamine,
(±)-{[7-(2,3-dimethoxyphenyl)-5-methyl-2,3-dihydro-1-benzofuran-2-yl]methyl}methylamine,
(±)-{[7-(2,4-dichlorophenyl)-5-methyl-2,3-dihydro-1-benzofuran-2-yl]methyl}methylamine,
(±)-{[7-(2,5-dichlorophenyl)-5-methyl-2,3-dihydro-1-benzofuran-2-yl]methyl}methylamine,
(+)-{[7-(2,5-dichlorophenyl)-5-methyl-2,3-dihydro-1-benzofuran-2-yl]methyl}methylamine,
(−)-{[7-(2,5-dichlorophenyl)-5-methyl-2,3-dihydro-1-benzofuran-2-yl]methyl}methylamine,
(±)-{[7-(2,6-dimethylphenyl)-5-methyl-2,3-dihydro-1-benzofuran-2-yl]methyl}methylamine,
(±)-{[7-(2,6-dichlorophenyl)-5-methyl-2,3-dihydro-1-benzofuran-2-yl]methyl}methylamine,
(±)-{[7-(2-fluorophenyl)-5-methoxy-2,3-dihydro-1-benzofuran-2-yl]methyl}methylamine,
(±)-{[7-(2-chlorophenyl)-5-methoxy-2,3-dihydro-1-benzofuran-2-yl]methyl}methylamine,
(±)-{[7-(2-methylphenyl)-5-methoxy-2,3-dihydro-1-benzofuran-2-yl]methyl}methylamine,
(±)-{[7-(2,3-difluorophenyl)-5-methoxy-2,3-dihydro-1-benzofuran-2-yl]methyl}methylamine,
(±)-{[7-(2,3-dichlorophenyl)-5-methoxy-2,3-dihydro-1-benzofuran-2-yl]methyl}methylamine,
(±)-{[7-(2,3-dimethylphenyl)-5-methoxy-2,3-dihydro-1-benzofuran-2-yl]methyl}methylamine,
(±)-{[7-(2,4-difluorophenyl)-5-methoxy-2,3-dihydro-1-benzofuran-2-yl]methyl}methylamine,
(±)-{[7-(2,5-difluorophenyl)-5-methoxy-2,3-dihydro-1-benzofuran-2-yl]methyl}methylamine,
(±)-{[7-(2,5-dichlorophenyl)-5-methoxy-2,3-dihydro-1-benzofuran-2-yl]methyl}methylamine,
(±)-{[7-(2,5-dimethylphenyl)-5-methoxy-2,3-dihydro-1-benzofuran-2-yl]methyl}methylamine,
(±)-{[7-(2,5-dimethoxyphenyl)-5-methoxy-2,3-dihydro-1-benzofuran-2-yl]methyl}methylamine,
(±)-{[7-(5-chloro-2-methoxyphenyl)-5-methoxy-2,3-dihydro-1-benzofuran-2-yl]methyl}methylamine,
(±)-{[7-(2,6-dimethylphenyl)-5-methoxy-2,3-dihydro-1-benzofuran-2-yl]methyl}methylamine,
(±)-N-methyl-1-[7-(2,3-difluorophenyl)-5-(trifluoromethyl)-2,3-dihydro-1-benzofuran-2-yl]methanamine,
(±)-N-methyl-1-[7-(3,4-difluorophenyl)-5-(trifluoromethyl)-2,3-dihydro-1-benzofuran-2-yl]methanamine,
(±)-N-methyl-1-[7-(2,5-difluorophenyl)-5-(trifluoromethyl)-2,3-dihydro-1-benzofuran-2-yl]methanamine,
(±)-N-methyl-1-[7-(2,3-dichlorophenyl)-5-(trifluoromethyl)-2,3-dihydro-1-benzofuran-2-yl]methanamine,
(±)-{[7-(3-chloro-4-fluorophenyl)-5-(trifluoromethyl)-2,3-dihydro-1-benzofuran-2-yl]methyl}methylamine,
(±)-N-methyl-1-[7-(2,4-dimethoxyphenyl)-5-(trifluoromethyl)-2,3-dihydro-1-benzofuran-2-yl]methanamine,
(+) {[7-(2,6-dichlorophenyl)-5-fluoro-2,3-dihydro-1-benzofuran-2-yl]methyl}methylamine,
(−) {[7-(2,6-dichlorophenyl)-5-fluoro-2,3-dihydro-1-benzofuran-2-yl]methyl}methylamine,
(R)-[7-(2-chloro-phenyl)-(5-Fluoro-2,3-dihydro-benzofuran-2-ylmethyl)methyl-amine,
(R)-[7-(2,6-dichloro-phenyl)-5-Fluoro-2,3-dihydro-benzofuran-2-ylmethyl]ethylamine,
(R)-[7-(2,6-dichloro-phenyl)-5-Fluoro-2,3-dihydro-benzofuran-2-ylmethyl]dimethylamine,
{[(2R)-7-(5-chloro-2-methylphenyl)-5-fluoro-2,3-dihydro-1-benzofuran-2-yl]methyl}amine,
{[(2R)-7-(4-chloro-2-methylphenyl)-5-fluoro-2,3-dihydro-1-benzofuran-2-yl]methyl}amine,
(−)-{[7-(2,6-dichlorophenyl)-5-fluoro-2,3-dihydro-1-benzofuran-2-yl]methyl}amine,
(+)-{[7-(2,6 dichlorophenyl)-5-fluoro-2,3-dihydro-1-benzofuran-2-yl]methyl}amine,
(±)-{2-[6-chloro-7-(2-chlorophenyl)-2,3-dihydro-1-benzofuran-2-yl]ethyl}amine,
(±)-{2-[7-(2,6-dichlorophenyl)-5-fluoro-2,3-dihydro-1-benzofuran-2-yl]ethyl}amine,
(±)-{2-[7-(2-methoxyphenyl)-5-methoxy-2,3-dihydro-1-benzofuran-2-yl]ethyl}amine,
(±)-{N-methyl-1-[(7-(2,4,6-trichlorophenyl)-2,3-dihydro-1-benzofuran-2-yl]methanamine,
(+)-{N-methyl-1-[(7-(2,4,6-trichlorophenyl)-2,3-dihydro-1-benzofuran-2-yl]methanamine,
(−)-{N-methyl-1-[(7-(2,4,6-trichlorophenyl)-2,3-dihydro-1-benzofuran-2-yl]methanamine,
(+)-{[7-(2,6-dimethylphenyl)-5-fluoro-2,3-dihydro-1-benzofuran-2-yl]methyl}methylamine,
(−)-{[7-(2,6-dimethylphenyl)-5-fluoro-2,3-dihydro-1-benzofuran-2-yl]methyl}methylamine,
(−)-{[7-(2,6-dimethylphenyl)-5-methoxy-2,3-dihydro-1-benzofuran-2-yl]methyl}methylamine,
(+)-{[7-(2,6-dimethylphenyl)-5-methoxy-2,3-dihydro-1-benzofuran-2-yl]methyl}methylamine,
(+)-{[5-chloro-7-(2,5-dichlorophenyl)-2,3-dihydro-1-benzofuran-2-yl]methyl}methylamine,
(−)-{[5-chloro-7-(2,5-dichlorophenyl)-2,3-dihydro-1-benzofuran-2-yl]methyl}methylamine,
(−)-{[5-chloro-7-(2,6-dimethylphenyl)-2,3-dihydro-1-benzofuran-2-yl]methyl}methylamine,
(+)-{[5-chloro-7-(2,6-dimethylphenyl)-2,3-dihydro-1-benzofuran-2-yl]methyl}methylamine,
(+)-{[7-(2,3-dimethoxyphenyl)-5-methyl-2,3-dihydro-1-benzofuran-2-yl]methyl}methylamine,
(−)-{[7-(2,3-dimethoxyphenyl)-5-methyl-2,3-dihydro-1-benzofuran-2-yl]methyl}methylamine,
(−)-{[7-(2,3-dimethoxyphenyl)-5-fluoro-2,3-dihydro-1-benzofuran-2-yl]methyl}methylamine,
(+)-{[7-(2,3-dimethoxyphenyl)-5-fluoro-2,3-dihydro-1-benzofuran-2-yl]methyl}methylamine,
or a pharmaceutically acceptable salt thereof.

17. The method of claim 1, wherein the urinary incontinence is urge incontinence.

18. The method of claim 17, wherein the urinary incontinence is secondary to prostate hypertrophy.

19. The method of claim 1, wherein the urinary incontinence is mixed urge and stress incontinence.

20. Use of a compound of formula I: 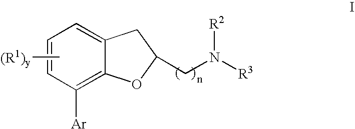
or a pharmaceutically acceptable salt thereof, wherein:
n is one or two;
each of R² and R³ is independently hydrogen, methyl, ethyl, 2-fluoroethyl, 2,2-difluoroethyl or cyclopropyl;
each R¹ is independently hydrogen, halogen, OH, lower alkyl, lower alkoxy, lower haloalkyl, lower haloalkoxy, or CN;
Ar is thienyl, furyl, pyridyl, or phenyl, wherein Ar is optionally substituted with one or more R^{x} subsituents;
each R^{x} is independently selected from halogen, OH, lower alkyl, lower alkoxy, lower haloalkyl, lower haloalkoxy, or CN; and
y is 0-3, for preparing a medicament for treating urinary incontinence in a mammal.
